(19) United States
(12) Patent Application Publication
(10) Pub. No.: US 2021/0093665 A1
(43) Pub. Date: Apr. 1, 2021

(54) CHIMERIC ANTIGEN RECEPTORS AGAINST SENESCENT CELLS AND USES THEREOF

(71) Applicant: Stark Labs, Lille (FR)

(72) Inventors: Müge Ogrunc, Paris (FR); Thierry Mathieu, Bruxelles (BE)

(73) Assignee: Stark Labs, Lille (FR)

(21) Appl. No.: 16/585,256

(22) Filed: Sep. 27, 2019

(51) Int. Cl. A61K 35/17; C07K 14/725; C07K 16/28

(52) U.S. Cl. CPC A61K 35/17; C07K 14/7051; C07K 2317/31; C07K 2319/03; C07K 2319/02; C07K 16/2896

(57) ABSTRACT

The invention relates to a chimeric antigen receptor (CAR) comprising directed to at least one senescent cell-associated antigen, preferably to DEP1 and/or DPP4; including bispecific CAR, bispecific antibodies, and isolated immune cell population expressing the same. It also relates to uses and methods for treating, preventing or alleviating senescence-related diseases or disorders, or for depleting and/or killing senescent cells.

### FIELD OF INVENTION

The invention relates to a chimeric antigen receptor (CAR) comprising directed to at least one senescent cell-associated antigen, preferably to DEP1 and/or DPP4; including bispecific CAR, bispecific antibodies, and isolated immune cell population expressing the same. It also relates to uses and methods for treating, preventing or alleviating senescence-related diseases or disorders, or for depleting and/or killing senescent cells.

### BACKGROUND OF INVENTION

Cellular senescence is an evolutionarily conserved state of stable replicative arrest induced by several pro-ageing stressors, including telomere attrition, oxidative stress, DNA damage and oncogene activation. Cellular senescence is associated with apoptosis resistance, and results in secretion of a broad repertoire of cytokines, chemokines, growth factors, matrix remodeling proteases: the so-called senescence-associated secretory phenotype (SASP). This cellular state also promotes proliferation and tissue deterioration.

Conversely, senescence is also anti-proliferative, and may be requisite for optimal cutaneous wound healing. Therefore, cellular senescence is an example of antagonistic pleiotropy in which natural selection favors processes that are beneficial early in life, even if they cause harmful effects later in post-reproduction life.

Since its discovery, senescence, once defined as the limited replicative capacity of primary human fibroblasts, now serves as a key player driving organismal aging via exhaustion of tissue repair capacity. Several human pathologies have been associated with detrimental effects of senescence such as lung fibrosis, type 2 diabetes, obesity, osteoarthritis, ocular diseases, Alzheimer's and Parkinson's disease (Munoz-Espin and Serrano, 2014. Nat Rev Mol Cell Biol. 2014 July; 15(7):482-96). Therapeutic strategies so far to balance these pathologies related to accumulation of senescent cells are dependent on direct elimination of senescent cells based on their intrinsic properties (e.g., their apoptotic resistance or P53 dependence) (Yosef et al., 2016. Nat Commun. 7:11190; Chang et al., 2016. Nat Med. 22(1):78-83; Baar et al., 2017. Cell. 169(1):132-147). Although these first-generation senolytic approaches serve as a proof-of-principle for drug discovery targeting senescence, they are limited by their observed toxic side effects.

While the role of senescence and the contribution of senescent cells are increasingly recognized in the context of aging and a variety of disease states, relatively little is known regarding the influences of senescent cells in normal lung growth and aging per se, or in the induction or progression of lung diseases across the age spectrum, such as bronchopulmonary dysplasia, asthma, chronic obstructive pulmonary disease, or pulmonary fibrosis. However, crucial evidences have been recently provided by several groups that cellular senescence contributes to lung ageing (Hashimoto et al., 2016. JCI Insight. 1(12):e87732; Lehmann et al., 2017. Eur Respir J. 50(2):1602367 ; Schafer et al., 2017. Nat Commun. 8:14532).

Among lung diseases, idiopathic pulmonary fibrosis (IPF) is a typical example of an ageing disease characterized by a progressive destruction of lung parenchyma and interstitial remodeling, leading to IPF symptoms (i.e., chronic shortness of breath, cough, fatigue and weight loss) and resulting in dramatic truncation of healthspan and lifespan.

The potential to blunt lung disease by targeting senescent cells using a novel class of drugs called “senolytics” is currently discussed. Indeed, two studies by Lehmann et al. and Schafer et al. suggest that cellular senescence is a salient feature of lung fibrosis, and that targeting/elimination of these cells could be beneficial. In particular, they show that cellular senescence markers such as SAβG, P21, P16INK4a and DNA damage response are detectable within IPF patients, as well as in experimental models of lung fibrosis. They further demonstrate that senescent cell elimination rejuvenates pulmonary health in aged mice. However, it is unclear whether and how senescent cells regulate IPF in humans or if their removal may be an efficacious intervention strategy.

Although promising, it cannot be excluded that senolytic drugs could be detrimental in IPF patients. Indeed, senolytic drug treatment may result in massive epithelial cell depletion by apoptosis, which could trigger diffuse alveolar damage and acute exacerbation, since the regenerative capacity of epithelial cells in IPF patients is impaired.

There remains thus a need for alternative strategies for depleting senescent cells and improving health and lung functions of IPF patients.

The Inventors have developed such alternative strategy, by potentiating an immune response against senescent cells in a way that would lead to their clearance from lung tissue. They provide herein a new association of two cell surface markers, DPP4 and DEP1, which are targeted to detect and deplete senescent cells in the lung. Senescent cells are immunogenic in nature and are subject to immune surveillance mechanisms.

Dipeptidyl peptidase 4 (DPP4, also named CD26) is a cell surface protease with a wide range of biological functions. As a serine-type protease, DPP4 preferentially cleaves off substrates with proline and alanine at the penultimate position. Expression of DPP4 is widespread throughout the body. Interestingly, DPP4 has been identified as a senescent cell surface targetable protein, functionally required for fibroblast activation and tissue fibrosis (Kyoung et al., 2017. Genes Dev. 31(15):1529-1534).

Density Enhanced Protein Tyrosine Phosphatase (DEP1, also named CD148, HPTP-eta, or PTP receptor type J (PTPRJ)) is an enzyme that removes phosphate groups covalently attached to tyrosine residues in proteins. DEP1 is highly expressed on both hematopoietic and nonhematopoietic cells, including lung cells. It has been shown that DEP1 can directly interact with and dephosphorylate the regulatory subunit of PI3K (p85) (Tsuboi et al., 2008. Biochem J. 413(1):193-200) and that hyperactivation of PI3K/Akt plays an important role in the profibrotic phenotype of IPF-derived lung fibroblasts by promoting cell proliferation and migration and myofibroblast differentiation (Kral et al., 2016. Sci Rep. 6:23034; Nho et al., 2014. PLoS One. 9(4):e94616).

The Inventors herein provide antibodies, bispecific antibodies, chimeric antigen receptors (CARs) and bispecific CARs, including immune cell populations expressing said CARs directed specifically against senescent cells for treatment and prophylaxis of age-related diseases and disorders, and other diseases and disorders associated or exacerbated by the presence of senescent cells, such as, for example, pulmonary fibrosis. The antibodies and CARs described herein are specific for at least one senescent cell-associated antigen (e.g., DEP-1 and/or DPP4), and induce the clearance (i.e., removal, elimination, destruction) of senescent cells. Said clearance may, for example, be mediated by antibody- dependent cell cytotoxicity (ADCC) or complement dependent cytotoxicity (CDC) or both.

### SUMMARY

The present invention relates to a chimeric antigen receptor (CAR) comprising:
(i) at least one extracellular binding domain, comprising at least one antigen-binding domain directed to a senescent cell-associated antigen, preferably to DEP1 and/or DPP4,
(ii) an extracellular spacer domain,
(iii) a transmembrane domain,
(iv) optionally at least one costimulatory domain, and
(v) at least one intracellular signaling domain.

In one embodiment, said at least one antigen-binding domain is directed to DEP1.

In one embodiment, said at least one antigen-binding domain is directed to DPP4.

In one embodiment, said CAR is a bispecific CAR comprising two antigen-binding domains.

In one embodiment, each of the at least two antigen-binding domains binds to a different antigen, preferably to DEP1 and DPP4.

The present invention also relates to an isolated immune cell population expressing at least one CAR according to the present invention, preferably the isolated immune cell population expresses:
at least one CAR directed to DEP1 and at least one CAR directed to DPP4; or
at least one bispecific CAR comprising two antigen-binding domains, preferably wherein each of the at least two antigen-binding domains binds to a different antigen, preferably to DEP1 and DPP4.

In one embodiment, the isolated immune cell population according to the present invention comprises immune cells selected from the group comprising T cells, natural killer (NK) cells, or a combination thereof.

The present invention also relates to an isolated bispecific antibody or a fragment thereof, comprising at least two antigen binding domains directed to at least two senescent cell-associated antigens, preferably the at least two senescent cell-associated antigens are DEP1 and DPP4.

In one embodiment, the isolated bispecific antibody or fragment thereof according to the present invention comprises:
(i) an antigen-binding domain of an anti-human DEP1 antibody or a fragment thereof and
(ii) an antigen-binding domain of an anti-human DPP4 antibody or a fragment thereof.

The present invention also relates to a composition comprising:
the isolated immune cell population according to the present invention;
the isolated bispecific antibody or fragment thereof according to the present invention; or
a mixture of an isolated anti-human DEP1 antibody or a fragment thereof and an isolated anti-human DPP4 antibody or a fragment thereof.

In one embodiment, the composition according to the present invention is a pharmaceutical composition and further comprises at least one pharmaceutically acceptable excipient.

In one embodiment, the composition or pharmaceutical composition according to the present invention is for use as a medicament.

In one embodiment, the composition or pharmaceutical composition according to the present invention is:
for use in treating, preventing or alleviating senescence-related diseases or disorders; or
for depleting and/or killing senescent cells.

In one embodiment, senescence-related diseases or disorders are selected from the group comprising fibrotic diseases, premalignant disorders, inflammatory diseases and cancers.

Definitions

“About”, preceding a figure encompasses plus or minus 10%, or less, of the value of said figure. It is to be understood that the value to which the term “about” refers is itself also specifically, and preferably, disclosed.

“Adnectins”, also known as monobodies, is well known in the art and refer to proteins designed to bind with high affinity and specificity to antigens. They belong to the class of molecules collectively called “antibody mimetics”.

“Allogeneic” refers to a graft derived from a different animal of the same species.

“Alphabody” that may also be referred to as Cell-Penetrating Alphabodies, refer to a type of antibody mimetics consisting of small 10 kDa proteins engineered to bind to a variety of antigens. Alphabodies are able to reach and bind to intracellular protein targets.

“Affibodies” are well-known in the art and refer to affinity proteins based on a 58 amino acid residue protein domain, derived from one of the IgG binding domain of staphylococcal protein A (Frejd & Kim, 2017. Exp Mol Med. 49(3):e306; U.S. Pat. No. 5,831,012).

“Affilins” are well known in the art and refer to artificial proteins designed to selectively bind antigens. They resemble antibodies in their affinity and specificity to antigens but not in structure which makes them a type of antibody mimetic “Affinity” and “avidity” are well-known in the art and are used to defined the strength of an antibody-antigen complex. Affinity measures the strength of interaction between an epitope and an antigen binding site on an antibody. It may be expressed by an affinity constant KA or by a dissociation constant KD. Avidity (or functional affinity) gives a measure of the overall strength of an antibody-antigen complex. It may depend on different parameters, including in particular the affinity of the antibody or antigen-binding fragment thereof for an epitope, (ii) the valency of both the antibody and the antigen and (iii) structural arrangement of the parts that interact. Affinities of antibodies or antigen-binding fragment thereof can be readily determined using conventional techniques, for example, those described by Scatchard, 1949. Ann NY Acad Sci. 51:660-672. Binding properties of an antibody or antigen-binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BlAcore®).

As used herein, the terms “antibody” and “immunoglobulin” may be used interchangeably and refer to a protein having a combination of two heavy and two light chains whether or not it possesses any relevant specific immunoreactivity. “Antibodies” refers to such assemblies which have significant known specific immunoreactive activity to an antigen of interest (e.g., human DEP-1 and/or DPP4). The term “anti-DEP-1 antibodies or anti-DPP4 antibodies” is used herein to refer to antibodies which exhibit immunological specificity for human DEP-1 antigen or human DPP4 antigens, respectively. As explained elsewhere herein, “specificity” for human DEP-1 does not exclude cross-reaction with species homologues of human DEP-1, such as, for example, with simian DEP-1, and “specificity” for human DPP4 does not exclude cross-reaction with species homologues of human DPP4 such as, for example, with simian DPP4.

Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (VL domain) and heavy chain variable domain (VH domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the VH and VL chains.

Anticalins” are well known in the art and refer to an antibody mimetic technology, wherein the binding specificity is derived from lipocalins. Anticalins may also be formatted as dual targeting protein, called Duocalins.

The term “Antigen” refers any substance that is capable of stimulating an immune response, specifically activating immunes cells. In general, two main divisions of antigens are recognized: foreign antigens (or heteroantigens) and autoantigens (or self-antigens). antigens). Antigen molecules possess by definition, at least one epitope (or antigenic sites) which produce corresponding antibodies.

The term “antigen-binding fragment”, as used herein, refers to a part or region of the antibody according to the present invention, which comprises fewer amino acid residues than the whole antibody. An “antigen-binding fragment” binds antigen and/or competes with the whole antibody from which it was derived for antigen binding (e.g., specific binding to human senescent associated-cell antigen). Antibody antigen-binding fragments encompasses, without any limitation, single chain antibodies, Fv, Fab, Fab′, Fab′-SH, F(ab)′2, Fd, defucosylated antibodies, diabodies, triabodies and tetrabodies.

“Armadillo repeat protein-based scaffold”, as used herein, refers to a type of antibody mimetics corresponding to artificial peptide binding scaffolds based on armadillo repeat proteins. Armadillo repeat proteins are characterized by an armadillo domain, composed of tandem armadillo repeats of approximately 42 amino acids, which mediates interactions with peptides or proteins.

“Atrimers” are well known in the art and refers to binding molecules for target protein that trimerize as a perquisite for their biological activity. They are relatively large compared to other antibody mimetic scaffolds.

As used herein, the term “autologous” is meant to refer to any material derived from the same individual to which it is later to be re-introduced into the individual.

“Avimers” are well known in the art and refer to an antibody mimetic technology.

As used herein, the term “CDR” or “complementarity determining region” means the non-contiguous antigen combining sites found within the variable region of both heavy and light chain polypeptides. The precise amino acid sequence boundaries of a given CDR can be determined using any of a number of well-known schemes, including those described by Kabat et al. (1991), “Sequences of Proteins of Immunological Interest” 5th Ed. Public Health Service, National Institutes of Health, Bethesda, M D (“Kabat” numbering scheme), Al-Lazikani et al., 1997. J Mol Biol. 273(4):927-48 (“Chothia” numbering scheme), or a combination thereof. More recently, a universal numbering system has been developed and widely adopted, ImMunoGeneTics (IMGT) Information System® (Lefranc et al., 1999. Nucleic Acids Res. 27(1):209-12). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues may be readily identified. This information can be used in grafting and replacement of CDR residues from immunoglobulins of one species into an acceptor framework from, typically, a human antibody. Correspondence between the Kabat numbering and the IMGT unique numbering system is also well known to one skilled in the art (e.g., Lefranc et al., supra). Thus, in one embodiment, by CDR regions or CDR, it is intended to indicate the hypervariable regions of the heavy and light chains of the immunoglobulins as defined by IMGT® numbering system (e.g. Lefranc et al., supra).

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid

“Chimeric antigen receptor” or “CAR” or “CARs” as used herein refers to engineered receptors, which graft an antigen specificity onto cells (such as, for example, T cells or phagocytic cells). CARs are also known as artificial T-cell receptors, chimeric T-cell receptors or chimeric immunoreceptors.

“Co-stimulatory domain” (CSD) as used herein refers to the portion of the CAR which enhances the proliferation, survival and/or development of memory cells. The CARs of the invention may comprise one or more co-stimulatory domains. Co-stimulatory domains are apparent to those of skill in the art and may be used in connection with alternate embodiments of the invention.

“DARPins” (Designed Ankyrin Repeat Proteins) are well known in the art and refer to an antibody mimetic DRP (designed repeat protein) technology developed to exploit the binding abilities of non-antibody polypeptides.

As used herein, the term “DEP1” (also known as PTPRJ, SCC1, CD148, HPTPeta or R-PTP-ETA) PTP-ETA) refers to a protein encoded by a gene which is a member of the protein tyrosine phosphatase (PTP) family. PTPs are known to be signaling molecules that regulate a variety of cellular processes, including cell growth, differentiation, mitotic cycle, and oncogenic transformation. This PTP possesses an extracellular region containing five fibronectin type III repeats, a single transmembrane region, and a single intracytoplasmic catalytic domain, and thus represents a receptor-type PTP. This protein is present in all hematopoietic lineages, and was shown to negatively regulate T cell receptor signaling possibly through interfering with the phosphorylation of Phospholipase C Gamma 1 and Linker for Activation of T Cells. This protein can also dephosphorylate the PDGF beta receptor, and may be involved in UV-induced signal transduction. In human, multiple transcript variants encoding different isoforms have been found for this gene.

“Diabodies”, as used herein, refers to small antibody fragments prepared by constructing scFv fragments with short linkers (about 5-10 residues) between the HCVR and LCVR such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the HCVR and LCVR of the two antibodies are present on different polypeptide chains. Diabodies are described more fully in European patent EP0404097, International patent application WO1993011161; and in Holliger et al., 1993. Proc Natl Acad Sci USA. 90(14):6444-8.

“Domain antibodies” are well-known in the art and refer to the smallest functional binding units of antibodies, corresponding to the variable regions of either the heavy or light chains of antibodies.

“Domain kunitz peptide” refer to a type of antibody mimetics, and is based on the active domains of proteins inhibiting the function of proteases.

As used herein, the term “DPP4” refers to a protein encoded by a gene which is identical to adenosine deaminase complexing protein-2 (ADCP2), and to the T-cell activation antigen CD26. DPP4 is an intrinsic membrane glycoprotein and a serine exopeptidase that cleaves X-proline dipeptides from the N-terminus of polypeptides.

As used herein, the term “epitope”, also known as antigenic determinant, refers to a specific arrangement of amino acids located on a protein or proteins (or antigen(s)) to which an antibody or antigen-binding fragment thereof binds. Epitopes often consist of a chemically active surface grouping of molecules such as amino acids or sugar side chains, and have specific three dimensional structural characteristics as well as specific charge characteristics. Epitopes can be linear (or sequential) or conformational, i.e., involving two or more sequences of amino acids in various regions of the antigen that may not necessarily be contiguous.

“Evasins” are well known in the art and refer to a class of chemokine-binding proteins.

“Extracellular spacer domain” (ESD) as used herein refers to the hydrophilic region which is between the antigen-specific targeting region and the transmembrane domain. The extracellular spacer domains are apparent to those of skill in the art and may be used in connection with alternate embodiments of the invention.

As used herein, the term “framework region” or “FR region” includes the amino acid residues that are part of the variable region, but are not part of the CDRs (e.g., using the IMGT® numbering definition of CDRs). The framework regions for the light chain are similarly separated by each of the LCVR's CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

The terms “Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof.

“Fynomers” are well known in the art and refer to proteins that belong to the class of antibody mimetic. They are attractive binding molecules due to their high thermal stability and reduced immunogenicity.

“Fv”, as used herein, refers to the minimum antibody fragment that contains a complete antigen-recognition and -binding site. This fragment consists of a dimer of one HCVR and one LCVR in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

As used herein, the term “heavy chain region” includes amino acid sequences derived from the constant domains of an immunoglobulin heavy chain. A protein comprising a heavy chain region comprises at least one of a C_{H}1 domain, a hinge (e.g., upper, middle, and/or lower hinge region) domain, a C_{H}2 domain, a C_{H}3 domain, or a variant or fragment thereof. In an embodiment, the antibody or antigen-binding fragment thereof according to the present invention may comprise the Fc region of an immunoglobulin heavy chain (e.g., a hinge portion, a C_{H}2 domain, and a C_{H}3 domain). In another embodiment, the antibody or antigen-binding fragment thereof according to the present invention lacks at least a region of a constant domain (e.g., all or part of a C_{H}2 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a protein may comprise a C_{H}2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the antibody or antigen-binding fragment thereof according to the present invention may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

As used herein, the term “hinge region” includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J Immunol. 161(8):4083-90).

The term “hypervariable loop” is not strictly synonymous to complementarity determining region (CDR), since the hypervariable loops (HVs) are defined on the basis of structure, whereas CDRs are defined based on sequence variability (Kabat et al., 1991. Sequences of proteins of immunological interest (5^{th} ed.).Bethesda, Md.: U.S. Dep. of Health and Human Services) and the limits of the HVs and the CDRs may be different in some V_{H} and V_{L} domains. The CDRs of the V_{L} and V_{H} domains can typically be defined by the Kabat/Chothia definition as already explained hereinabove.

As used herein, the term “identity” or “identical”, when used in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Such methods include, but are not limited to, those described in Lesk A. M. (1988). Computational molecular biology: Sources and methods for sequence analysis. New York, N.Y.: Oxford University Press; Smith D. W. (1993). Biocomputing: Informatics and genome projects. San Diego, Calif.: Academic Press; Griffin A. M. & Griffin H. G. (1994). Computer analysis of sequence data, Part 1. Totowa, N.J.: Humana Press; von Heijne G. (1987). Sequence analysis in molecular biology: treasure trove or trivial pursuit. San Diego, Calif.: Academic press; Gribskov M. R. & Devereux J. (1991). Sequence analysis primer. New York, N.Y.: Stockton Press; Carillo et al., 1988. SIAM J Appl Math. 48(5):1073-82. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, Wis.; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Intracellular signaling domain” (ISD) or “cytoplasmic domain” as used herein refer to the portion of the CAR which transduces the effector function signal and directs the cell to perform its specialized function. Intracellular signaling domains are be apparent to those of skill in the art and may be used in connection with alternate embodiments of the invention.

“Isolated” means altered or removed from the natural state. For example, a nucleic acid or a peptide naturally present in a living animal is not “isolated” but the same nucleic acid or peptide partially or completely separated from the coexisting materials of its natural state is “isolated”. An isolated nucleic acid or protein can exist in substantially purified form, or can exist in a non-native environment such as, for example, a host cell.

“Knottin” (that may also be referred to as inhibitor cystine not) refer to an antibody mimetic comprising a protein structural motif containing three disulfide bridges.

As used herein, the term “monoclonal antibody” refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies or antigen-binding fragment thereof according to the present invention may be prepared by the hybridoma methodology first described by Kohler et al., 1975. Nature. 256(5517):495-7, or may be made using recombinant DNA methods in bacterial, eukaryotic animal or plant cells (U.S. Pat. No. 4,816,567). The “monoclonal antibodies” may also be isolated from phage antibody libraries using the techniques described in Clackson et al., 1991. Nature. 352(6336):624-8 and Marks et al., 1991. J Mol Biol. 222(3):581-97, for example.

“Linker” (L) as used herein refer to an oligo- or polypeptide region from about 1 to 100 amino acids in length, which links together any of the domains/regions of the CAR of the invention. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Linkers are apparent to those of skill in the art and may be used in connection with alternate embodiments of the invention.

“Nanobodies” are well-known in the art and refer to antibody-derived therapeutic proteins that contain the unique structural and functional properties of naturally-occurring heavy chain antibodies (Muyldermans, 2013. Annu Rev Biochem. 82:775-97). These heavy chain antibodies may contain a single variable domain (VHH) and two constant domains (C_{H}2 and C_{H}3).

As used herein, the terms “prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

As used herein, the term “proliferating cell” refers to a cell that is undergoing cell division.

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding (Plückthun, 1994. “Antibodies from Escherichia coli”. In Rosenberg & Moore (Eds.), The pharmacology of monoclonal antibodies. Handbook of Experimental Pharmacology, 113:269-315. Springer: Berlin, Heidelberg).

As used herein, the term “subject” refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human.

The term “therapeutically effective amount” refers to the level or amount of an antibody as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid.

“Transmembrane domain” (TMD) as used herein refers to the region of the CAR which crosses the plasma membrane. The transmembrane domain of the CAR of the invention is the transmembrane region of a transmembrane protein (for example Type I transmembrane proteins), an artificial hydrophobic sequence or a combination thereof. Other transmembrane domains are apparent to those of skill in the art and may be used in connection with alternate embodiments of the invention.

As used herein, the term “treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a cancer or an infection if, after receiving a therapeutic amount of an antibody according to the methods of the present invention, the patient shows observable and/or measurable reduction in or absence of one or more of the following: reduction in the number of cancer cells (or tumor size), or pathogenic cells; reduction in the percent of total cells that are cancerous or pathogenic; and/or relief to some extent, one or more of the symptoms associated with the specific disease or condition; reduced morbidity and mortality, and improvement in quality of life issues. The above parameters for assessing successful treatment and improvement in the disease are readily measurable by routine procedures familiar to a physician.

“Unibodies” are well known in the art and refer to an antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies.

As used herein, the term “variable” refers to the fact that certain regions of the variable domains V_{H} and V_{L} differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the V_{L} domain and the V_{H} domain which form part of the antigen binding site. The first, second and third hypervariable loops of the Vλ light chain domain are referred to herein as L1(λ), L2 (λ) and L3 (λ) and may be defined as comprising residues 24-33 (L1(λ), consisting of 9, 10 or 11 amino acid residues), 49-53 L2 (λ), consisting of 3 residues) and 90-96 (L3(λ), consisting of 6 residues) in the V_{L} domain (Morea et al., 2000. Methods. 20(3):267-79). The first, second and third hypervariable loops of the Vκ light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the V_{L} domain (Morea et al., 2000. Methods. 20(3):267-79). The first, second and third hypervariable loops of the V_{H} domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., 2000. Methods. 20(3):267-79). Unless otherwise indicated, the terms L1, L2 and L3 respectively refer to the first, second and third hypervariable loops of a VL domain, and encompass hypervariable loops obtained from both Vκ and Vλ isotypes. The terms H1, H2 and H3 respectively refer to the first, second and third hypervariable loops of the V_{H} domain, and encompass hypervariable loops obtained from any of the known heavy chain isotypes, including gamma (γ), mu (μ), alpha (α), delta (δ) or epsilon (ε). The hypervariable loops L1, L2, L3, H1, H2 and H3 may each comprise part of a “complementarity determining region” or “CDR”, as defined hereinabove.

“Versabodies” are well known in the art and refer to another antibody mimetic technology. They are small proteins of 3-5 kDa with >15% cysteines, which form a high disulfide density scaffold, replacing the hydrophobic core the typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MEW presentation are hydrophobic. All four of these properties are well-known to affect immunogenicity, and together they are expected to cause a large decrease in immunogenicity.

“Xenogeneic” refers to a graft derived from an animal of a different species.

### DETAILED DESCRIPTION

A first object of the present invention is a chimeric antigen receptor (CAR) comprising:
(i) at least one extracellular binding domain, comprising at least one antigen-binding domain directed to a senescent cell-associated antigen, preferably to DEP1 and/or DPP4,
(ii) an extracellular spacer,
(iii) a transmembrane domain,
(iv) at least one costimulatory domain, and
(v) at least one intracellular signaling domain.

In one embodiment, the CAR of the invention comprises one or more polypeptides.

In one embodiment, the CAR of the invention recognizes and is capable to bind to a senescent cell-associated antigen.

As used herein, the term “senescent cells” refers to cells that are in cell cycle arrest, generally during the G1 transition of the cell cycle or in few cases in G2, elicited by replicative exhaustion due to telomere attrition or in response to stresses such as DNA damage, chemotherapeutic drugs, or aberrant expression of oncogenes. According to one embodiment, the senescent cells are generally characterized by at least one or more of the following characteristics: activation of the p53/p21CIP1 and/or pRb/p16INK4A tumor suppressor pathways, cells whose proliferation is irreversibly arrested, shortening of telomere size, expression of senescent-associated beta-galactosidase activity, specific chromatin modification, specific secretome, increase in reactive oxygen species and altered overall mitochondrial activity. Senescent cells and senescent cell associated antigens can be detected by techniques and procedures described in the art.

The presence of senescent cells can be determined by detection of senescent cell-associated molecules include growth factors, proteases, cytokines (e.g., inflammatory cytokines), chemokines, cell-related metabolites, reactive oxygen species (e.g., H₂O₂), and other molecules that stimulate inflammation and/or other biological effects or reactions that may promote or exacerbate the underlying disease of the subject. Senescent cell-associated molecules include those that are described in the art as comprising the senescence-associated secretory phenotype (SASP, i.e., which includes secreted factors which may make up the pro-inflammatory phenotype of a senescent cell), senescent-messaging secretome, and DNA damage secretory program (DDSP). For example, the presence of senescent cells in tissues can be analyzed by histochemistry or immunohistochemistry techniques that detect the senescence marker, SA-beta gal (SA-Bgal) (see, for exemple, Dimri et al., 1995. Proc Natl Acad Sci USA. 92(20):9363-7).

Senescent cell-associated antigens include molecules that are overexpressed in senescent cells compared to their quiescent or non-senescent counterparts. Certain senescent cell-associated antigens are tissue specific while others are ubiquitously overexpressed in senescent cells. In particular embodiments of the immunogenic compositions described herein, a senescent cell-associated antigen is an antigen present on the cell surface of a senescent cell (e.g., receptor proteins, channel forming proteins, proteins that facilitate diffusion or active transport of molecules and ion across the membrane, cell recognition proteins, and enzymes). These antigens may be present on the cell surface of a cell exclusively or at a greater level on senescent cells compared with non-senescent cells and are therefore useful as immunogens for evoking a specific immune response. Examples of senescent cell-associated antigens include polypeptides and proteins (including glycoproteins), lipids, glycolipids, and carbohydrate molecules that contribute to or are markers of a senescence cell.

Thus, in one embodiment, the term “senescent cells” also refers to cells which express a senescent cell-associated antigen or a combination of senescent cell-associated antigens that are characteristic of senescence. Such senescent cell-associated antigens include, but are not limited to, actin cytoplasmic 1 (ACTB), A disintegrin and metalloproteinase with thrombospondin motifs 7 (ADAMTS7), amyloid-like protein 2 (APLP2), armadillo repeat-containing X-linked protein 3 (ARMCX-3), ATP synthase subunit alpha mitochondrial (ATP5F1A), V-type proton ATPase subunit d 2 (ATP6V0D2), beta-2-microglobulin (B2MG), cholinesterase (BCHE), uncharacterized protein C1lorf87 (C1lorf87), membrane cofactor protein (CD46), CD57, cyclin-dependent kinase inhibitor 2A “p16INK4a” (CDKN2A), cathepsin B (CTSB), neuferricin (CYB5D2), dipeptidyl peptidase 4 “DPP4” (DPP4), electron transfer flavoprotein beta subunit lysine methyltransferase (ETFB), F-box/LRR-repeat protein 7 (FBXL7), integral membrane protein GPR137B (GPR137B), interferon alpha-inducible protein 27-like protein 1 (IFI27L1), interleukin-15 receptor subunit alpha (IL15RA), killer cell lectin-like receptor subfamily G member 1 (KLRG1), lysosome-associated membrane glycoprotein 2 (LAMP2), glutathione S-transferase LANCL1 (LANCL1), major vault protein (MVP), unconventional myosin-X (MYOID), sialidase-1 (NEU1), NETS-like protein 2 (NHSL2), neurogenic locus notch homolog protein 3 (NOTCH3), neuronal PAS domain-containing protein 2 (NPAS2), olfactory receptor 1F1 (OR1F1), prolyl 4-hydroxylase beta subunit precursor (P4HB), protein disulfide isomerase (PDI), astrocytic phosphoprotein PEA-15 (PEA15), phospholipase D3 (PLD3), receptor-type tyrosine-protein phosphatase C isoform RA “CD45RA” (PTPRC), receptor-type tyrosine-protein phosphatase eta “DEP-1” (PTPRJ), Ras-related protein Rab-23 (RAB23), retinoic acid receptor beta (RARB), RNA-binding region-containing protein 3 (RNPC3), protein adenylyltransferase SelO mitochondrial (SELO), thioredoxin reductase-like selenoprotein T (SELT), semaphorin-5B (SEMA5B), stress-associated endoplasmic reticulum protein 1 (SERP1), plasminogen activator inhibitor 1 (SERPINE1), sodium/hydrogen exchanger 7 (SLC9A7), sorting nexin-3 (SNX3), syntaxin-4 (STX4), TBC1 domain family member 1 (TBC1D1), transforming growth factor beta regulator 1 (TBRG1), transcription elongation factor A N-terminal and central domain-containing protein (TCEANC), tissue factor pathway inhibitor (TFPI), BTB/POZ domain-containing adapter for CUL3-mediated RhoA degradation protein 2 (TNFAIP1), tumor necrosis factor receptor superfamily member 10D “DCR2” (TNFRSF10D), tubulin gamma-2 chain (TUBG2), Ubl carboxyl-terminal hydrolase 18 (USP18), vesicle-associated membrane protein 3 (VAMP3), vacuolar protein sorting-associated protein 26A (VPS26A), and zinc finger protein 419 (ZNF419).

In one embodiment, the senescent cell-associated antigen is selected from receptor-type tyrosine-protein phosphatase eta “DEP-1” and dipeptidyl peptidase 4 “DPP4”.

In one embodiment, the senescent cells express DEP-1 and/or DPP4 antigens.

The presence of the senescent cell-associated antigens, in particular of senescent cell-associated DEP-1 and/or DPP4 antigens, can be determined by any one of numerous immunochemistry methods practiced in the art, such as immunoblotting analysis

In one embodiment, the senescent cell-associated antigen is DEP-1, for example, murine, human, or rat DEP-1. In one embodiment, the senescent cell-associated antigen is DPP4, for example, murine, human, or rat DPP4. Thus, in one embodiment, the senescent cells express the DEP1 and/or DPP4 antigen.

In one embodiment, the CAR of the invention recognizes and is capable to bind to DEP1. Human DEP1 is a protein encoded by an mRNA comprising 27 exons (Genbank accession number: NM_002843).

In one embodiment, the CAR of the invention recognizes and is capable to bind to a DEP1 variant, preferably a variant of a human DEP1.

In one embodiment, the CAR of the invention recognizes and is capable to bind to DPP4. Human DPP4 is a protein encoded by an mRNA comprising 26 exons (Genbank accession number: NM_001935).

In one embodiment, the CAR of the invention recognizes and is capable to bind to a DPP4 variant, preferably a variant of a human DPP4.

In one embodiment, the extracellular binding domain is antigen-binding domain directed to a senescent cell-associated antigen, as defined hereinabove.

In one embodiment, the extracellular binding domain is antigen-binding domain directed to DEP1. In one embodiment, the antigen-binding domain is an antibody directed to DEP1 or an antigen binding fragment thereof.

In one embodiment, the extracellular binding domain is an antigen-binding domain directed to DPP4. In another embodiment, the antigen-binding domain is an antibody directed to DPP4 or an antigen binding fragment thereof.

The portion of the CAR of the invention comprising an antibody or antigen binding fragment thereof may exist in a variety of forms where the antigen binding domain is expressed as part of a contiguous polypeptide chain including, for example, a single chain antibody (scFv), a single domain antibody fragment (sdAb), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988. Proc Natl Acad Sci USA. 85(16):5879-83; Bird et al., 1988. Science. 242(4877):423-6).

The portion of the CAR of the invention comprising an antibody can be any type of immunoglobulin that is known in the art. For instance, the antibody can be of any isotype (e.g., IgA, IgD, IgE, IgG, IgM, etc). The portion of the CAR of the invention comprising an antibody can be monoclonal or polyclonal. The portion of the CAR of the invention comprising an antibody can be a naturally-occurring antibody, (e.g., an antibody isolated and/or purified from a mammal), (e.g., mouse, rabbit, goat, horse, chicken, hamster, human, etc). Alternatively, the portion of the CAR of the invention comprising an antibody can be a genetically-engineered antibody (e.g., a humanized antibody or a chimeric antibody). The portion of the CAR of the invention comprising an antibody can be in monomeric or polymeric form. Also, the antibody can have any level of affinity or avidity for the functional portion of the inventive CAR.

In one embodiment, the CAR of the invention comprises a whole antibody, a single chain antibody, a dimeric single chain antibody, a Fv, a scFv, a Fab, a F(ab)′₂, a defucosylated antibody, a bi-specific antibody, a diabody, a triabody, a tetrabody, a unibody, a domain antibody, a nanobody, or an antigen-binding fragment thereof.

In one embodiment, the extracellular binding domain is an antibody mimetic. Examples of antibody mimetics include, but are not limited to, an affibody, an alphabody, an armadillo repeat protein based scaffold, a knottin, a kunitz domain peptide, an affilin, an affitin, an adnectin, an atrimer, an evasin, a DARPin, an anticalin, an avimer, a fynomer, a versabody or a duocalin.

In one embodiment, the extracellular binding domain of the CAR of the invention comprises or consists in an antibody fragment, such as, for example, a scFv. In one embodiment, the scFv bind to a senescent cells surface molecule. In one embodiment, the scFv bind to DEP1. In another embodiment, the scFv bind to DPP4.

In one embodiment, the CAR comprises or consists of a scFv comprising a heavy chain V_{H} and a light chain V_{L}.

In one embodiment, the scFv comprises a linker that links the V_{H} and the V_{L} chains.

In one embodiment, the antibody comprised in the CAR of the invention is a multispecific antibody molecule, e.g., it comprises a plurality of immunoglobulin variable domain sequences, wherein a first immunoglobulin variable domain sequence of the plurality has binding specificity for a first epitope (or antigen) and a second immunoglobulin variable domain sequence of the plurality has binding specificity for a second epitope (or antigen). In one embodiment, a multispecific antibody molecule is a bispecific antibody molecule. A bispecific antibody has specificity for no more than two antigens. A bispecific antibody can distinctly recognize two antigens simultaneously. A bispecific antibody molecule is characterized by a first antigen binding domain which has binding specificity for a first epitope (or antigen) and a second antigen binding domain that has binding specificity for a second epitope (or antigen).

According to one embodiment, the CAR of the invention is a bispecific CAR. In this design the bispecific chimeric antigen receptor comprises (i) two antigen-binding domain wherein each antigen-binding domains binds a different antigen, and wherein each of the at least two antigen-binding domains binds to a different senescent cell-associated antigen, preferably to DEP1 and DPP4 antigens, (ii) an extracellular spacer domain, (iii) a transmembrane domain, (iv) at least one costimulatory domain, and (v) at least one intracellular signaling domain.

In one embodiment, the bispecific CAR comprises two extracellular binding domain, wherein the first extracellular antigen-binding domain binds to DEP1 and wherein the second extracellular antigen-binding domain binds to DPP4. Therefore, according to this embodiment, the CAR of the invention is capable of binding DEP1 and DPP4 antigens.

In a bispecific CAR, the antigen-binding domain may be the Fab fragment of an antibody or the single chain variable fragment (scFv) of an antibody. Thus, in one embodiment, the extracellular binding domain of the bispecific CAR as described herein above comprises two distinct scFv fragments directed against DEP-1 and DPP4 antigens.

In one embodiment, the bispecific CAR is a tandem CAR. Bispecific tandem CAR has already been described (see, for example, International application: WO2013123061, US. Patent application: US20130280220). Thus according to one embodiment, the two extracellular antigen-binding domain are configured in the bispecific CAR in a tandem arrangement. in one embodiment, there is a linker between the two non-identical antigen-binding domains, such as, for example, a linker region being between 5 and 30 amino acids. The linker region may be comprised of glycine, serine, or both.

in one embodiment, the design of the tandem CAR incorporates, in tandem. (i) an extracellular-binding domain, comprising two antigen-binding domain, wherein the two antigen-binding domain are single chain antibody variable fragments (scFv) tethered to a hinge, (ii) an extracellular spacer domain, (iii) a transmembrane domain, (iv) at least one costimulatory domain and (v) at least one signaling domain. Thus, the tandem CAR, has not one but two recognition domains, in tandem, projecting outside the immune cells enabling a single immune cell to recognize two molecules. Thus, in one embodiment, the extracellular binding domain of the tandem CAR as described herein above comprises two distinct scFv fragments directed against two different senescent cell-associated antigens, preferably DEP-1 and DPP4 antigens.

In one embodiment, the CAR is a hi specific CAR and targets two different antigens. As described above, the antigen-specific targeting regions of the CAR may be arranged in tandem and may be separated by linker peptides. The antigens targeted by the CAR may be antigens on single diseased cell or antigens that are expressed on separate cells that each contribute to the disease. The antigens targeted by the CAR. are antigens which are either directly or indirectly involved in the disease.

In another embodiment, the extracellular binding domain is connected to a transmembrane domain by an extracellular spacer or hinge domain.

In one embodiment, extracellular spacer or hinge domain facilitates proper protein folding. The extracellular spacer domain comprises a hydrophilic region which is attached to the antigen-specific targeting region and the transmembrane domain. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer or hinge domains include but are not limited to CD8α hinge, CD28 hinge, artificial spacers made of polypeptides such as Gly3, or CH1, CH3 domains of IgG's (such as human IgG4). Specifically, the extracellular spacer domain may be (i) a hinge, CH2 and CH3 regions of IgG4, (ii) a hinge region of IgG4, (iii) a hinge and CH2 of IgG4, (iv) a hinge region of CD8α, (v) a hinge, CH2 and CH3 regions of IgG1, (vi) a hinge region of IgG1 or (vii) a hinge and CH2 of IgG1, (viii) a hinge region of CD28 or a combination thereof. Generally the extracellular spacer or hinge domain is a short oligo- or polypeptide linker, having a length ranging from 2 to 10 amino acids. Additional extracellular spacer domains will be apparent to those of skill in the art and may be used in connection with alternate embodiments of the invention.

In another embodiment, the transmembrane domain comprises the transmembrane sequence from any protein which has a transmembrane domain, including any of the type I, type II or type III transmembrane proteins. The transmembrane domain may also comprise an artificial hydrophobic sequence. Examples of transmembrane domains that may be used in the chimeric receptor of the invention include, but are not limited to, transmembrane domains of an alpha, beta or zeta chain of a T-cell receptor, or of CD28, CD3 gamma, CD3 delta, CD3 epsilon, CD3 zeta, CD45, CD4, CD5, CD8, CD9, CD16, CD22, CD33, CD37, CD64, CD80, CD86, CD134, CD137, CD154, KIRDS2, OX40, CD2, CD27, LFA-1 (CD1 1a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, PD1, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR,

PAG/Cbp, NKp44, NKp30, NKp46, NKG2D, and/or NKG2C. Additional transmembrane domains will be apparent to those of skill in the art and may be used in connection with alternate embodiments of the invention.

In one embodiment, the transmembrane domain may be recombinant, in which case it will comprise predominantly hydrophobic amino acids such as valine or leucine.

In another embodiment, the costimulatory domain enhances cell proliferation, cell survival and development of memory cells. The CARs of the invention may comprise one or more costimulatory domains. Each costimulatory domain comprises the costimulatory domain of any one or more of, for example, members of the TNFR super family, CD28, CD137 (4-1BB), CD134 (OX40), Dap10, CD27, CD2, CD5, ICAM-1, Lck, TNFR-1, TNFR-II, Fas, CD30, CD40, CTLA-4, ICOS, PD-1 or combinations thereof. Costimulatory domains from other proteins may also be used with the CARs of the invention. Additional costimulatory domains will be apparent to those of skill in the art and may be used in connection with alternate embodiments of the invention. If a CAR comprises more than one co-stimulatory domain, these domains may be arranged in tandem, optionally separated by a linker.

In one embodiment, the costimulatory domain comprises a T cell costimulatory molecule (or a sequence derived therefrom).

In one embodiment of the invention, the CAR comprises at least one costimulatory domain selected from the group comprising 4-1BB, ICOS, CD27, OX40, CD28, CTLA4 and PD-1.

In one embodiment, the at least one intracellular signaling domain is cytoplasmic and transduce the effector function signal and direct the cell to perform its specialized function. The CARs of the invention may comprise one or more intracellular signaling domains. Examples of intracellular signaling domains include, but are not limited to, ζ chain of the T-cell receptor or any of its homologs (e.g., η chain, FcεR1γ and β chains. MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

Specifically, the intracellular signaling domain may be human CD3 zeta chain, FcγRIII, FcεRI, cytoplasmic tails of Fc receptors, immunoreceptor tyrosine-based activation motif (ITAM) bearing cytoplasmic receptors or combinations thereof. Additional intracellular signaling domains will be apparent to those of skill in the art and may be used in connection with alternate embodiments of the invention.

In one embodiment, the intracellular signaling domain may comprise the entire intracellular portion, or the entire native intracellular signaling domain, of the molecule from which it is derived, or a functional fragment or derivative thereof.

In one embodiment, the intracellular signaling domain comprises a T cell primary signaling domain (or a sequence derived therefrom).

In one embodiment of the invention, the T cell primary signaling domain comprises a signaling domain of a protein selected in the group of CD3 zeta, CD3 gamma, CD3 delta, CD3 epsilon, common FcR gamma (FCER1G), FcR beta (Fc Epsilon Rib), CD79a, CD79b, Fcgamma RIIa, DAP 10, and DAP 12 and sequences derived therefrom.

In one embodiment, the T cell primary signaling domain comprises or consists in a functional signaling domain of CD3 zeta.

T cell primary signaling domains that act in a stimulatory manner may comprise signaling motifs known as immunoreceptor tyrosine-based activation motifs (ITAMS).

Examples of ITAM containing T cell primary intracellular signaling domains that are of particular use in the invention include, but are not limited to, those of (or derived from) CD3 zeta, common FcR gamma (FCER1G), Fc gamma RIIa, FcR beta (Fc Epsilon R1b), CD3 gamma, CD3 delta, CD3 epsilon, CD5, CD22, CD66b, CD79a, CD79b, DAP10, and DAP12.

In one embodiment, the T cell primary signaling domain comprises a modified ITAM domain, (e.g., a mutated ITAM domain which has altered e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

In one embodiment, the primary signaling domain and the at least one costimulatory domain (e.g., T cell costimulatory molecule) may be linked to each other in tandem, in a random or in a specified order.

Optionally, a short oligo- or polypeptide linker, for example, between 2 and 10 amino acids (e.g., 2, 3, 4, 5, 6, 7, 8, 9, or 10 amino acids) in length may form the linkage between distinct signaling domains. In one embodiment, a glycine-serine doublet (GS) is used as a suitable linker. In one embodiment, a single amino acid, e.g., an alanine (A), a glycine (G), is used as a suitable linker. Other examples of linker are described herein.

In one embodiment, the intracellular signaling domain of the CAR of the invention comprises two or more, e.g., 2, 3, 4, 5, or more, intracellular signaling domains.

In another embodiment, the two or more, e.g., 2, 3, 4, 5, or more, intracellular signaling domains, are separated by a linker molecule, e.g., a linker molecule as described hereinabove.

In one embodiment, the CAR further comprises a tag, such as, for example, a tag for quality control, enrichment, tracking in vivo and the like. Said tag may be localized N-terminally, C-terminally and/or internally. Examples of tags that may be used in the CAR of the invention are well known by the skilled artisan. For example, but without limitation, a tag used in the invention can be a tag selected from the group comprising or consisting of Hemagglutinin Tag, Poly Arginine Tag, Poly Histidine Tag, Myc Tag, Strep Tag, S-Tag, HAT Tag, 3x Flag Tag, Calmodulin-binding peptide Tag, SBP Tag, Chitin binding domain Tag, GST Tag, Maltose-Binding protein Tag, Fluorescent Protein Tag, T7 Tag, V5 Tag and Xpress Tag.

The CAR of the invention may be a first generation, second generation, third generation or fourth generation CAR.

According to one embodiment, the CAR of the invention is a first generation CAR and comprises (i) an extracellular binding domain, (ii) an extracellular hinge domain, (iii) a transmembrane domain, and (iv) an intracellular signaling domain. A first generation CAR can be, for example, a CAR in which signaling is provided by CD3.

According to one embodiment, the CAR of the invention is a second generation CAR and comprises (i) an extracellular binding domain, (ii) an extracellular hinge domain, (iii) a transmembrane domain, (iv) a costimulatory domain, and (v) an intracellular signaling domain.

According to one embodiment, the CAR of the invention is a third generation CAR and comprises (i) an extracellular binding domain, (ii) an extracellular hinge domain, (iii) a transmembrane domain, (iv) at least two costimulatory domain, and (v) an intracellular signaling domain.

According to one embodiment, the CAR of the invention is part of a fourth generation CAR-T cell which comprises a CAR with (i) an extracellular binding domain, (ii) an extracellular hinge domain, (iii) a transmembrane domain, (iv) at least two costimulatory domain, (v) an intracellular signaling domain; and a transgene coding for an antibody, a bispecific antibody, a cytokine or a costimulatory ligand.

As used herein, the “fourth generation” CAR cells are defined as CAR cells armed with an antibody, a bispecific antibody (including BiTE), an immune stimulatory cytokines or a costimulatory ligand that improve CAR cell expansion and persistence. In one embodiment, the antibody, bispecific antibody, immune stimulatory cytokines or costimulatory ligand gene is activated when CAR signal activates immune cells.

In one embodiment, the fourth generation CAR conditionally or constitutively secretes antibodies or bispecific antibodies (including BiTE) as described hereinafter.

In one embodiment, the fourth generation CAR conditionally or constitutively secretes cytokines, such as, for example, IL-2, IL-12, IL-15 and IL-18.

In one embodiment, the fourth generation CAR conditionally or constitutively secretes costimulatory ligands, such as, for example, GITR ligand (GITRL), CD27 ligand (CD70) and ICOS ligand (B7h or LICOS), HVEM ligand (LIGHT), OX40 ligand (OX40L), 4-1BB ligand (4-1BBL or CD137L) or CD28 ligand (B7-1/CD80, B7-2/CD86).

The present invention further relates to immune cells, preferably isolated immune cells engineered to express at the cell surface a CAR or a bispecific CAR as described hereinabove.

The present invention also relates to an isolated and/or substantially purified immune cell population comprising cells engineered to express at the cell surface a CAR or a bispecific CAR as described hereinabove.

In one embodiment, the immune cells of the invention express a CAR at their cell surface, wherein the extracellular domain (or antigen binding domain) of the CAR recognizes a senescent cell-associated antigen, as defined hereinabove.

In one embodiment, the immune cells of the invention express a CAR at their cell surface, wherein the extracellular domain (or antigen binding domain) of the CAR recognizes DEP1.

In one embodiment, the immune cells of the invention express a CAR at their cell surface, wherein the extracellular domain (or antigen binding domain) of the CAR recognizes DPP4.

In one embodiment, the immune cells of the invention express at least two CARs at their cell surface, wherein the first CAR and the second CAR recognize two different senescent cell-associated antigens.

In one embodiment, the immune cells of the invention express at least two CARs at their cell surface, wherein the first CAR recognizes DEP1, and the second CAR recognizes DPP4.

In one embodiment, the immune cells of the invention express a bispecific CAR at their cell surface, wherein the extracellular domain of the bispecific CAR comprises two antigen binding domains, and wherein the first antigen binding domain recognizes DEP1 and the second antigen binding domain recognizes DPP4.

The present invention also relates to an isolated and/or substantially purified immune cell population comprising cells engineered to express at the cell surface a CAR as described hereinabove.

The present invention also relates to an immune cell engineered to express: (a) at least one CAR or a bispecific CAR as described herein above, and (b) a bispecific T cell engager (BiTE), wherein the BiTE binds to a senescent cell-associated antigen as defined hereinabove and a T cell antigen as defined hereinabove. For example, described herein are immune cells, e.g., T cells engineered to express a CAR as well as to secrete a bispecific T cell engager (BiTE). Of note, CAR T cells engineered to secrete BiTEs are referred to herein as CAR-T BiTE.

In one embodiment, the immune cells are T cells, preferably isolated T cells. In one embodiment, the immune cells of the invention comprises or consists in T cells. In one embodiment, the T cells comprises or consists in CD8⁺ T cells, CD4⁺ T cells, natural killer (NK) cells and NKT cells or a combination thereof.

In one embodiment, the T cells are cytotoxic T cell (also known as TC, Cytotoxic T Lymphocyte, CTL, T-Killer cell, cytolytic T cell, CD8⁺ T-cells or killer T cell), NK cells and NKT cells are also encompassed in the invention.

In one embodiment, the T cell is a CD8⁺ T cytotoxic lymphocyte cell selected from the group consisting of naive CD8⁺ T cells, CD8⁺ memory T cells, central memory CD8⁺ T cells, regulatory CD8⁺ T cells, IPS derived CD8⁺ T cells, effector memory CD8⁺ T cells and bulk CD8⁺ T cells. In some alternatives, the T cell is a CD4⁺ T helper lymphocyte cell that is selected from the group consisting of naive CD4⁺ T cells, CD4⁺ memory T cells, central memory CD4⁺ T cells, regulatory CD4⁺ T cells, IPS derived CD4⁺ T cells, effector memory CD4⁺ T cells and bulk CD4⁺ T cells. Thus, in one embodiment, the immune cells of the invention are cytotoxic for cells expressing at their surface the DEP1 and/or DPP4 antigens recognized by the CAR. In one embodiment, the T cells of the invention are not cytotoxic for cells expressing at their surface the DEP1 and/or DPP4 antigen recognized by the CAR.

In one embodiment, the immune cells of the invention are phagocytic cells.

In one embodiment, the phagocytic cells are selected from the group consisting of macrophage, monocyte, histiocyte, Kupffer cell, alveolar macrophage, microglial cell, dendritic cell and others.

In one embodiment, the immune cells as described herein are mammal cells, preferably human immune cells.

In one embodiment, the immune cells as described herein can be autologous cells, syngeneic cells, allogenic cells, and even in some cases, xenogeneic cells.

Prior to expansion and genetic modification of the immune cells of the invention (e.g., T cells or phagocytic cells), a source of immune cells (e.g., T cells or phagocytic cells) are/is obtained from a subject. Thus, in one embodiment, the immune cells or immune cell population of the invention are/is isolated and/or substantially purified

T cells and/or phagocytic cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell and/or phagocytic cells lines available in the art, may be used. In certain embodiments of the present invention, T cells and/or phagocytic cells can be obtained from a unit of blood collected from a subject using any number of techniques known to the skilled artisan, such as FicollTM separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, cells from the circulating blood of an individual are obtained by leukapheresis.

In one embodiment, the cells collected by leukapheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the leukapheresis sample may be removed and the cells directly resuspended in culture media.

In another embodiment, T cells and/or phagocytic cells are isolated from peripheral blood lymphocytes by lysing the red blood cells and depleting the monocytes, for example, by centrifugation through a PERCOLL™ gradient or by counterflow centrifugal elutriation. A specific subpopulation of T cells can be further isolated by positive or negative selection techniques. The skilled artisan would recognize that multiple rounds of selection can also be used in the context of this invention.

In another embodiment, it may be desirable to perform the selection procedure and use the “unselected” cells in the activation and expansion process. “Unselected” cells can also be subjected to further rounds of selection. Enrichment of a T cell population by negative selection can be accomplished with a combination of antibodies directed to surface markers unique to the negatively selected cells. One method is cell sorting and/or selection via negative magnetic immuno-adherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, T regulatory cells are depleted by anti-CD25 conjugated beads or other similar method of selection. Another example, to enrich monocyte, macrophage and/or dendritic cell population by negative selection, a monoclonal antibody cocktail typically includes antibodies to CD34, CD3, CD4, CD8, CD14, CD19 or CD20.

In certain embodiments, cryopreserved cells are thawed and washed as described herein and allowed to rest for one hour at room temperature prior to activation.

Also contemplated in the context of the invention is the collection of blood samples or leukapheresis product from a subject at a time period prior to when the expanded cells as described herein might be needed. As such, the source of the cells to be expanded can be collected at any time point necessary, and desired cells, such as T cells and/or phagocytic cells, isolated and frozen for later use in cell therapy for any number of diseases or conditions that would benefit from cell therapy, such as those described herein. In one embodiment, a blood sample or a leukapheresis is taken from a generally healthy subject. In certain embodiments, a blood sample or a leukapheresis is taken from a generally healthy subject who is at risk of developing a disease, but who has not yet developed a disease, and the cells of interest are isolated and frozen for later use. In certain embodiments, the T cells and/or phagocytic cells may be expanded, frozen, and used at a later time.

In one embodiment, the immune cells or population of immune cells of the invention are cultured for expansion. In another embodiment, the immune cells or population of immune cells of the invention comprising progenitor cells are cultured for differentiation and expansion of the immune cells or population of immune cells as described herein.

Whether prior to or after genetic modification of the immune cells (i.e., T cells or phagocytic cells) to express a desirable CAR or bispecific CAR as described herein, the immune cells (i.e., T cells and/or phagocytic cells) can be activated and expanded generally using methods as described, for example, in U.S. Pat. No. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US20060121005, incorporated herein by reference.

In one embodiment, the immune cell (i.e., T cell or phagocytic cell) population as described herein is a genetically modified immune cells (i.e., T cells or phagocytic cell). In one embodiment, the genetically modified immune cells (i.e., T cells) of the invention can be an allogeneic immune cells (i.e., T cells or phagocytic cell). For example, the allogeneic immune cell can be an immune cell lacking expression of a functional human leukocyte antigen (HLA), e.g., HLA class I and/or HLA class II or a T-cell receptor (TCR).

In one embodiment, the immune cells as described herein can be engineered such that they do not express a functional HLA and/or TCR on its surface. For example, an immune cell as described herein can be engineered such that cell surface expression HLA, e.g., HLA class 1 and/or HLA class II or non-classical HLA molecules is downregulated.

Modified immune cells that lack expression of a functional HLA and/or TCR can be obtained by any suitable means, including a knock out or knock down of one or more subunit of HLA. For example, the immune cell can include a knock down of HLA using siRNA, shRNA, clustered regularly interspaced short palindromic repeats (CRISPR) transcription-activator like effector nuclease (TALEN), zinc finger endonuclease (ZFN), meganuclease (mn, also known as homing endonuclease), or megaTAL (combining a TAL effector with a mn cleavage domain). Such system are well known in the art.

In one embodiment, the nucleic acid encoding a CAR as described herein is inserted at a specific locus in the genome of an immune cell, such as, for example, at the locus of a gene to be deleted. In one embodiment, the nucleic acid encoding a CAR as described herein is inserted within a HLA locus, thereby resulting in the inhibition of HLA expression.

In one embodiment, the CAR of the invention when expressed by a T cell or phagocytic cell, confers to the T cell or phagocytic cell the ability to bind to cells expressing DEP1 and/or DPP4 on their cell surface and be activated by DEP1 and/or DPP4, differently from the antigen that the T cells or phagocytic cell are or would have been specific or activated by.

The T cell or phagocytic cell population of the invention may thus be defined as a redirected T cell or phagocytic cell population. As used herein, the term “redirected” refers to a T cell or phagocytic cell carrying a chimeric receptor as described herein, which confers to the T cell or phagocytic cell, the ability to bind to and be activated by a ligand that is different from the one the T cell or phagocytic cell is or would have been specific or be activated by.

In one embodiment, the genetically modified immune cells (i.e., T cells or phagocytic cell) of the invention can express certain gene products that can kill the modified cells under controlled conditions, such as inducible suicide genes.

The present invention further relates to a nucleic acid sequence encoding a CAR as described hereinabove.

In one embodiment, the present invention encompasses a DNA construct comprising sequences of a CAR, wherein the sequence comprises the nucleic acid sequence of an antigen binding domain (i.e., senescent cell-associated binding domain) operably linked to the nucleic acid sequence of transmembrane domain and a cytoplasmic domain (e.g., at least one costimulatory domain and/or at least one intracellular signaling domain).

In one embodiment, the nucleic acid sequence encoding a CAR of the present invention comprises (i) a nucleic acid sequence of an extracellular senescent cell-associated binding domain, preferably DEP1 or DPP4 binding domain, (ii) a nucleic acid sequence of an extracellular spacer domain, (iii) a nucleic acid sequence of a transmembrane domain, (iv) at least one nucleic acid sequence of a costimulatory domain and (v) at least one nucleic acid sequence of a intracellular signaling domain and (vi) optionally a nucleic acid sequence of a Tag and or a leader sequence.

In one embodiment, the nucleic acid sequence encoding a bispecific CAR of the present invention comprises (i) a nucleic acid sequence of an extracellular binding domain comprising two antigen-binding domains that binds to senescent cell, preferably to DEP1 and DPP4, (ii) a nucleic acid sequence of an extracellular spacer domain, (iii) a nucleic acid sequence of a transmembrane domain, (iv) optionally at least one nucleic acid sequence of a costimulatory domain and (v) at least one nucleic acid sequence of a intracellular signaling domain and (vi) optionally a nucleic acid sequence of a Tag and or a leader sequence.

The nucleic acid encoding a CAR or a bispecific CAR of the invention can be prepared in conventional ways (e.g., recombinant methods), where the genes and regulatory regions may be isolated, as appropriate, ligated, and cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. Alternatively, the gene of interest can be produced synthetically, rather than cloned.

Another object of the invention is a vector comprising the nucleic acid sequence encoding a CAR or a bispecific CAR as described hereinabove. Indeed, the CAR or a bispecific CAR according to the invention may be expressed from an expression vector. Recombinant techniques to generate such expression vectors are well known in the art.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

Another object of the invention is a vector comprising a first nucleic acid sequence encoding a CAR or a bispecific CAR as described hereinabove and, optionally, a second nucleic acid sequence encoding an antibody, a bispecific antibody (e.g., a BiTE), a cytokine or a costimulatory ligand. In one embodiment, the first nucleic acid sequence and the second nucleic acid sequence are each operably linked to a promoter. In another embodiment, the first nucleic acid sequence is operably linked to a first promoter and the second nucleic acid sequence is operably linked to a second promoter. The promoter can be a constitutively expressed promoter (e.g., an EF1 a promoter) or an inducibly expressed promoter (e.g., a NFAT promoter).

In some embodiments, expression of the CAR and expression of the antibody, the bispecific antibody (e.g., a BiTE), the cytokine or the costimulatory ligand are driven by the same promoter, e.g., a constitutively expressed promoter (e.g., an EF1 a promoter). In other embodiments, expression of the CAR and expression of the antibody, the bispecific antibody (e.g., a BiTE), the cytokine or the costimulatory ligand are driven by different promoters.

In one embodiment, the nucleic acid sequence encoding the CAR can be located upstream of the nucleic acid sequence encoding the the antibody, the bispecific antibody (e.g., a BiTE), the cytokine or the costimulatory ligand; or can be located upstream the nucleic acid sequence encoding the CAR.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned” “operatively linked” “under control” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5 prime' non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring”, i.e., containing different elements of different transcriptional regulatory regions, and/or mutations that alter expression. For example, promoters that are most commonly used in recombinant DNA construction include the lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages, and these may be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™ 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 hours, the cells are collected by centrifugation and washed to remove residual media.

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present invention may be a viral vector that encodes one or more CARs, or/and bispecific CAR of the invention. Non-limiting examples of virus vectors that may be used to deliver a nucleic acid of the present invention are described below.

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

The nucleic acid may be introduced into the cell using adenovirus assisted transfection. Increased transfection efficiencies have been reported in cell systems using adenovirus coupled systems (Kelleher & Vos, 1994. Biotechniques. 17(6):1110-7; Cotten et al., 1992. Proc Natl Acad Sci USA. 89(13):6094-8; Curiel, 1994. Nat Immun. 13(2-3):141-64). Adeno associated virus (AAV) is an attractive vector system for use in the cells of the present invention as it has a high frequency of integration and it can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

Retroviruses are useful as delivery vectors because of their ability to integrate their genes into the host genome, transferring a large amount of foreign genetic material, infecting a broad spectrum of species and cell types and of being packaged in special cell lines.

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed. When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Other viral vectors may be employed as vaccine constructs in the present invention. Vectors derived from viruses such as vaccinia virus, sindbis virus, cytomegalovirus and herpes simplex virus may be employed. They offer several attractive features for various mammalian cells.

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin. Using antibodies against major histocompatibility complex class I and class II antigens, they demonstrated the infection of a variety of human cells that bore those surface antigens with an ecotropic virus in vitro.

Suitable methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

Thus, the present invention further relates to a method for obtaining an immune cell of the invention, wherein said method comprises transducing at least one immune cell with a nucleic acid encoding a CAR as described hereinabove, and optionally expanding the transduced cells.

Methods for transfecting eukaryotic cells and tissues removed from an organism in an ex vivo method are known to those of skill in the art. In one embodiment, the method is an ex vivo method. Thus, it is contemplated that cells or tissues may be removed and transfected ex vivo using nucleic acids of the present invention. In one embodiment, the transplanted cells or tissues may be placed into an organism. In one embodiment, a nucleic acid is expressed in the transplanted immune cell population.

The nucleic acid encoding a CAR or a bispecific CAR of the invention once completed and demonstrated to have the appropriate sequences may then be introduced into the immune cell by any convenient means. Said nucleic acid sequence may be introduced by fusion, electroporation, biolistics, transfection, lipofection, or the like. Alternatively, said nucleic acid sequence may also be integrated and packaged into non-replicating, defective viral genomes like adenovirus, adeno-associated virus (AAV), or herpes simplex virus (HSV) or others, including retroviral vectors or lentiviral vectors, for infection or transduction into cells. The nucleic acid encoding a CAR or a bispecific CAR may include viral sequences for transfection, if desired. The engineered cells may be grown and expanded in culture before introduction of the construct(s), followed by the appropriate treatment for introduction of the construct(s) and integration of the construct(s). The engineered cells are then expanded and screened by virtue of a marker present in the construct. Various markers that may be used successfully include hprt, neomycin resistance, thymidine kinase, hygromycin resistance, etc.

In one embodiment, one may have a target site for homologous recombination, where it is desired that a nucleic acid be integrated at a particular locus. For example, can knock-out an endogenous gene and replace it (at the same locus or elsewhere) with the gene encoded for by the construct using materials and methods as are known in the art for homologous recombination. For homologous recombination, one may use either OMEGA or O-vectors. See, for example, Thomas & Capecchi, 1987. Cell. 51(3):503-12; Mansour et al., 1988. Nature. 336(6197):348-352; and Joyner et al., 1989. Nature. 338(6211):153-156.

The nucleic acid encoding a CAR or a bispecific CAR may be introduced as a single DNA molecule encoding at least the CAR or bispecific CAR according to the invention and optionally another gene, or different DNA molecules having one or more genes. Other genes include genes that encode therapeutic molecules or suicide genes, for example. The constructs may be introduced simultaneously or consecutively, each with the same or different markers.

In one embodiment of the invention, suicide gene technology may be used. Different suicide gene technologies are described in the art depending on their mechanism of action (Jones et al., 2014. Front Pharmacol. 5:254). Examples of gene-directed enzyme prodrug therapy (GDEPT) converting a nontoxic drug to a toxic drug include herpes simplex virus thymidine kinase (HSV-TK) and cytosine deaminase (CD). Other examples are chimeric proteins composed of a drug binding domain linked to apoptotic components such as for example the inducible Fas (iFas) or the inducible Caspase 9 (iCasp9) systems. Other examples include systems mediated by therapeutic antibodies such as inducing overexpression of c-myc at the surface of the engineered cell to induce their deletion by administration of an anti-c-myc antibody. The use of EGFR is described as a similar system compared to the c-myc system.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

In one embodiment, the method for obtaining immune cells (e.g., T cells) of the invention comprises:
an isolation step of immune cells (e.g., T cells) from a PBMC population (e.g., recovered by leukapheresis)
a genetic modification step wherein a nucleic acid sequence encoding a CAR as described hereinabove is introduced or transferred within the immune cells (e.g., T cells),
optionally an expansion step,
optionally a washing step and,
optionally a freezing step.

In one embodiment, the genetic modification step(s) correspond(s) to a gene disruption step, a gene correction step or a gene addition step, preferably a gene addition step. In one embodiment, the genetic modification step(s) is carried out by a method selected from the group comprising, but not limited to, transfection, transduction or gene editing.

Examples of methods of gene editing that may be used in the present invention include, but are not limited to, methods based on engineered nucleases, methods based on recombinant Adeno-Associated Virus (or AAV), methods based on Transposons (e.g., Sleeping Beauty transposon system), methods based on homologous recombination, conditional targeting using site-specific recombinases (e.g., Cre-LoxP and Flp-FRT systems), and Multiplex Automated Genomic Engineering (MAGE).

Non-limiting examples of engineered nucleases include, but are not limited to, clustered regularly interspaced short palindromic repeats (CRISPR) transcription-activator like effector nuclease (TALEN), zinc finger endonuclease (ZFN), meganuclease (mn, also known as homing endonuclease), or megaTAL (combining a TAL effector with a mn cleavage domain).

In one embodiment, the method for obtaining immune cells (e.g., T cells) of the invention comprises:
an isolation step of immune cells (e.g., T cells) from a PBMC population (e.g., recovered by leukapheresis)
a transduction or transfection step with a vector comprising a nucleic acid sequence encoding a CAR as described hereinabove,
optionally an expansion step,
optionally a washing step and,
optionally a freezing step.

The immune cells (e.g., T cells or phagocytic cells) that have been modified with the nucleic acid encoding a CAR or a bispecific CAR of the invention are then grown in culture under selective conditions and cells that are selected as having the construct may then be expanded and further analyzed, using, for example; the polymerase chain reaction for determining the presence of the construct in the host cells. Once the modified host cells have been identified, they may then be used as planned (e.g. expanded in culture or introduced into a host organism).

Another object of the invention is an isolated antibody or antigen-binding fragment thereof, wherein said antibody or antigen-binding fragment thereof binds to a senescent cell-associated antigen, preferably to DEP-1 and/or DPP4 antigen.

In one embodiment, isolated antibody or antigen-binding fragment thereof according to the present invention binds to DEP-1.

In one embodiment, isolated antibody or antigen-binding fragment thereof according to the present invention binds to DPP4.

In one embodiment, the antibody or antigen-binding fragment thereof according to the present invention is specific for senescent cell-associated antigens, preferably for human DEP-1 and/or human DPP4 antigens.

An antibody or antigen-binding fragment thereof is said to be “specific for”, “immunospecific” or to “specifically bind” an antigen if it reacts with said antigen (e.g., DEP-1 and/or DPP4). An antibody or antigen-binding fragment thereof is said to be “immunospecific”, “specific for” or to “specifically bind” an antigen if it reacts at a detectable level with said antigen (e.g., DEP-1 and/or DPP4), preferably with a K_{D} of less than or equal to 10⁻⁶ M, preferably less than or equal to 10⁻⁷ M, 5.10⁻⁸ M, 10⁻⁸ M, 5.10⁻⁹ M, 10⁻⁹ M or less.

In one embodiment, the antibody or antigen-binding fragment thereof according to the present invention is a molecule selected from the group comprising or consisting of a whole antibody, a humanized antibody, a single chain antibody, a dimeric single chain antibody, a Fv, a Fab, a Fab′, a Fab′-SH, a F(ab)′₂, a Fd, a defucosylated antibody, a bi-specific antibody, a diabody, a triabody and a tetrabody.

In one embodiment, the antibody or antigen-binding fragment thereof according to the present invention is a mimetic selected from the group comprising or consisting of an affibody, an alphabody, an armadillo repeat protein based scaffold, a knottin, a kunitz domain peptide, an affilin, an affitin, an adnectin, an atrimer, an evasin, a DARPin, an anticalin, an avimer, a fynomer, a versabody or a duocalin.

Antigen-binding fragment that contain specific binding sites may be generated by a known method. Methods for producing antigen-binding fragments of antibodies are known in the art, for example as described in Lo (Ed.), 2004. Antibody Engineering: Methods and Protocols (1^{st} ed., Vol. 248). Totowa, N.J.: Humana Press; and McCafferty, Hoogenboom & Chiswell (Eds.), 1996. Antibody Engineering: a Practical Approach (1^{st} ed., Vol. 169). Oxford: IRL Press at Oxford University Press. For example, F(ab′)₂ fragments can be produced by pepsin digestion of the antibody molecule, and Fab fragments can be generated by reducing the disulfide bridges of the F(ab′)₂ fragments. Alternatively, Fab expression libraries may be constructed to allow rapid and easy identification of monoclonal Fab fragments with the desired specificity, as described for example in Huse et al., 1989. Science. 246(4935):1275-81, herein incorporated by reference.

Antibodies may be generated using known methods. For the production of antibodies, various hosts including goats, rabbits, rats, mice, humans, and others, may be immunized by injection with an appropriate antigen, Depending on the host species, various adjuvants may be used to increase an immunological response. Such adjuvants include Freund's adjuvant, mineral gels such as aluminium hydroxide, and surface-active substances such as lysolecithin, pluronic polyols, polyanions, peptides, oil emulsions, keyhole limpet hemocyanin, and dinitrophenol. Adjuvants are commercially available.

In one embodiment, the antibody or antigen-binding fragment thereof is a bispecific antibody with antigen binding to at least two senescent cell-associated antigens, and wherein the bispecific antibody binds to a different senescent cell-associated antigen as described herein above. In one embodiment, the bispecific antibody binds to DEP-1 and DPP4.

In one embodiment, the bispecific antigen is a bispecific T cell engager (BiTE).

By “bispecific T cell engager” or “BiTE” is meant a polypeptide that includes tandemly linked single-chain variable fragments (scFvs). Optionally, the scFvs are linked by a linker (e.g., a glycine-rich linker). One scFv of the BiTE binds to the T cell receptor (e.g., to the CD3c subunit) and the other binds to a senescent cell-associated antigen (e.g., a DEP1 or DPP4 antigen). In one embodiment, the BiTE binds to (i) a senescent cell-associated antigen as described hereinabove, and (ii) a T cell antigen.

Methods for producing bispecific antibodies (including BiTE) are also well known in the art.

In one embodiment, the antibody or antigen-binding fragment thereof according to the present invention is polyclonal. In another embodiment, the antibody or antigen-binding fragment thereof according to the present invention is monoclonal.

Methods of producing polyclonal and monoclonal antibodies as well as fragments thereof are well known in the art (see, for example, Harlow and Lane, Antibodies: A Laboratory Manual, Cold Spring Harbor Laboratory, New York, 1988).

In one embodiment, the antibody or antigen-binding fragment thereof according to the present invention comprises an isolated antibody. Methods for producing and isolating antibodies are well known in the art.

In one embodiment, the antibody or antigen-binding fragment thereof according to the present invention is a purified antibody or a purified antigen-binding fragment thereof according to the present invention.

In one embodiment, the antibody or antigen-binding fragment thereof according to the present invention has an isotype selected from the group consisting of IgG1, IgG2a, IgG3, IgG3 and IgA.

The constant region of an antibody is important in the ability of an antibody to fix complement and mediate cell-dependent cytotoxicity and phagocytosis. Thus, as discussed herein, the isotype of an antibody may be selected on the basis of whether it is desirable for the antibody to mediate cytotoxicity/phagocytosis. Determination or selection of the isotype of an antibody may be by known methods in the art.

In one embodiment, the antibody or antigen-binding fragment thereof according to the present invention is a fully or substantially murine antibody or fragment thereof

In one embodiment, the constant region is of murine origin.

In one embodiment, the antibody or antigen-binding fragment thereof according to the present invention is a murine antibody or fragment thereof.

In one embodiment, the antibody or antigen-binding fragment thereof according to the present invention is a chimeric antibody or fragment thereof.

A “chimeric antibody”, as used herein, refers to an antibody or antigen-binding fragment thereof comprising a first amino acid sequence linked to a second amino acid sequence with which it is not naturally linked in nature. The amino acid sequences may normally exist in separate proteins that are brought together in the fusion protein or they may normally exist in the same protein but are placed in a new arrangement in the fusion protein. A chimeric protein may be created, for example, by chemical synthesis, or by creating and translating a polynucleotide in which the peptide regions are encoded in the desired relationship. The term “chimeric antibody” encompasses herein antibodies and antigen-binding fragment thereof in which (a) the constant region, or a portion thereof, is altered, replaced or exchanged so that the variable region is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass. Method to produce chimeric antibodies are well known in the art For example, chimeric antibodies may be produced as described in Morrison et al., 1984. Proc Natl Acad Sci USA 81(21):6851-5, Neuberger et al., 1984. Nature. 312(5995):604-608; and Takeda et al., 1985. Nature. 314(6014452-454.

In one embodiment, the antibody or antigen-binding fragment thereof according to the present invention is a humanized antibody or fragment thereof.

A “humanized antibody”, as used herein, refers to a chimeric antibody or antigen-binding fragment thereof which contains minimal sequence derived from a non-human immunoglobulin. It includes antibodies made by a non-human cell having variable and constant regions which have been altered to more closely resemble antibodies that would be made by a human cell, e.g., by altering the non-human antibody amino acid sequence to incorporate amino acids found in human germline immunoglobulin sequences. Humanized antibodies or antigen-binding fragment thereof according to the present invention may include amino acid residues not encoded by human germline immunoglobulin sequences (e.g., mutations introduced by random or site-specific mutagenesis in vitro or by somatic mutation in vivo), for example in the CDRs. The term “humanized antibody” also includes antibodies and antigen-binding fragment thereof in which CDR sequences derived from the germline of another mammalian species, such as a mouse, have been grafted onto human framework sequences. In other words, the term “humanized antibody” refers to an antibody or antigen-binding fragment thereof in which the CDRs of a recipient human antibody are replaced by CDRs from a donor non-human antibody. Humanized antibodies or antigen-binding fragments thereof may also comprise residues of donor origin in the framework sequences. The humanized antibody or antigen-binding fragment thereof can also comprise at least a portion of a human immunoglobulin constant region. Humanized antibodies and or antigen-binding fragments thereof may also comprise residues which are found neither in the recipient antibody nor in the imported CDR or framework sequences. Humanization can be performed using methods known in the art (e.g., Jones et al., 1986. Nature. 321(6069):522-5; Riechmann et al., 1988. Nature. 332(6162):323-7; Verhoeyen et al., 1988. Science. 239(4847):1534-6; Presta, 1992.Curr Opin Biotechnol. 3(4):394-8; U.S. Pat. No. 4,816,567), including techniques such as “superhumanizing” antibodies (e.g., Tan et al., 2002. J Immunol. 169(2):1119-25) and “resurfacing” (e.g., Staelens et al., 2006. Mot Immunol. 43(8):1243-57; Roguska et al., 1994. Proc Natl Acad Sci USA. 91(3):969-73).

A “humanized antibody” retains a similar antigenic specificity as the original antibody. However, using certain methods of humanization, the affinity and/or specificity of binding of the antibody may be increased.

Methods for humanizing the antibody or antigen-binding fragment thereof according to the present invention are well-known in the art. The choice of human variable domains, both light and heavy, to be used in making the humanized antibody or antigen-binding fragment thereof is very important to reduce antigenicity. According to the so-called “best-fit” method, the sequence of the variable domain of an antibody or antigen-binding fragment thereof according to the present invention is screened against the entire library of known human variable-domain sequences. The human sequence that is closest to the mouse sequence is then accepted as the human framework (FR) for the humanized antibody (Sims et al., 1993. J Immunol. 151(4):2296-308; Chothia & Lesk, 1987. J Mol Biol. 196(4):901-17).

Another method for humanizing the antibody or antigen-binding fragment thereof according to the present invention uses a particular framework from the consensus sequence of all human antibodies of a particular subgroup of light or heavy chains. The same framework can be used for several different humanized antibodies (Carter et al., 1992. Proc Natl Acad Sci USA. 89(10):4285-9; Presta et al., 1993. J Immunol. 151(5):2623-32). It is further important that antibodies be humanized with retention of high affinity for hCD25 and other favorable biological properties. To achieve this goal, according to a preferred method, humanized antibodies and antigen-binding fragments thereof are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its epitope. In this way, CDR residues can be selected and combined from the consensus and import sequences so that the desired antibody characteristic, such as an increased affinity for hCD25, is achieved. In general, the CDR residues are directly and most substantially involved in influencing antigen binding.

Another method for humanizing the antibody or antigen-binding fragment thereof according to the present invention is to use a transgenic or transchromosomic animal carrying parts of the human immune system for immunization. As a host, these animals have had their immunoglobulin genes replaced by functional human immunoglobulin genes. Thus, antibodies produced by these animals or in hybridomas made from the B cells of these animals are already humanized. Examples of such transgenic or transchromosomic animal include, without limitation:
the XenoMouse (Abgenix, Fremont, Calif.), described in U.S. Pat. Nos. 5,939,598, 6,075,181, 6,114,598, 6,150,584 and 6,162,963;
the HuMAb Mouse® (Medarex, Inc.), described in Lonberg et al., 1994. Nature.
368(6474):856-859; Lonberg & Huszar, 1995. Int Rev Immunol. 13(1):65-93; Harding & Lonberg, 1995. Ann N Y Acad Sci. 764:536-46; Taylor et al., 1992. Nucleic Acids Res. 20(23):6287-95; Chen et al., 1993. Int Immunol. 5(6):647-56; Tuaillon et al., 1993. Proc Natl Acad Sci USA. 90(8):3720-4; Choi et al., 1993. Nat Genet. 4(2):117-23; Chen et al., 1993. EMBO J. 12(3):821-30; Tuaillon et al., 1994. J Immunol. 152(6):2912-20; Taylor et al., 1994. Int Immunol. 6(4):579-91; Fishwild et al., 1996. Nat Biotechnol. 14(7):845-51;
the KM Mouse®, described in Patent application WO2002043478;
the TC mice, described in Tomizuka et al., 2000. Proc Natl Acad Sci USA. 97(2):722-7; and
the OmniRat™ (OMT, Inc.), described in Patent application WO2008151081; Geurts et al., 2009. Science. 325(5939):433; Menoret et al., 2010. Eur J Immunol. 40(10):2932-41.

Humanized antibodies and antigen-binding fragments thereof may also be produced according to various other techniques, such as by using, for immunization, other transgenic animals that have been engineered to express a human antibody repertoire (Jakobovitz et al., 1993. Nature. 362(6417):255-8), or by selection of antibody repertoires using phage display methods. Such techniques are known to the skilled person and can be implemented starting from monoclonal antibodies or antigen-binding fragments thereof as disclosed in the present application.

In one embodiment, the antibody or antigen-binding fragment thereof according to the present invention is a fully or substantially human antibody or fragment thereof.

In one embodiment, the constant region is of human origin.

In some embodiment, especially when the antibody or antigen-binding fragment thereof according to the present invention is intended for human therapeutic uses, it is typical for the entire constant region, or at least a part thereof, to have a fully or substantially human amino acid sequence. Therefore, one or more of, or any combination of, the C_{H}1 domain, hinge region, C_{H}2 domain, C_{H}3 domain and C_{L} domain (and C_{H}4 domain if present) may be fully or substantially human with respect to its amino acid sequence. Advantageously, the C_{H}1 domain, hinge region, C_{H}2 domain, C_{H}3 domain and C_{L} domain (and C_{H}4 domain if present) may all have a fully or substantially human amino acid sequence.

The term “substantially human”, in the context of the constant region of a humanized or chimeric antibody or antigen-binding fragment thereof, refers to an amino acid sequence identity of at least 70%, preferably at least 75%, 80%, 85%, 90%, 95%, 96%, 97%, 98%, 99% or more with a human constant region.

The term “human amino acid sequence”, in this context, refers to an amino acid sequence which is encoded by a human immunoglobulin gene, which includes germline, rearranged and somatically mutated genes. The present invention also contemplates proteins comprising constant domains of “human” sequence which have been altered, by one or more amino acid additions, deletions or substitutions with respect to the human sequence, excepting those embodiments where the presence of a “fully human hinge region” is expressly required.

The presence of a “fully human hinge region” in the antibody or antigen-binding fragment thereof according to the present invention may be beneficial both to minimize immunogenicity and to optimize stability of the antibody. It is considered that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g., by addition or deletion of N- or O-linked glycosylation sites). Depending on the intended use of the antibody or antigen-binding fragment thereof, it may be desirable to modify the antibody or antigen-binding fragment thereof according to the present invention with respect to its binding properties to Fc receptors, for example to modulate effector function. For example, cysteine residue(s) may be introduced in the Fc region, thereby allowing interchain disulfide bond formation in this region. The homodimeric antibody thus generated may have improved effector function (Caron et al., 1992. J Exp Med. 176(4):1191-5; Shopes, 1992. J Immunol. 148(9):2918-22).

In one embodiment, the antibody or antigen-binding fragment thereof according to the present invention is a recombinant antibody or fragment thereof.

Thus accordingly, the antibody or antigen-binding fragment thereof according to the present invention may also be produced recombinantly by methods known in the art, such as, for example by expression in E.coii expression systems (see, far example, the U.S. Pat. No. 4,816,567). Antigen binding fragment may also be produced by phage display technologies, which are known in the art.

It will also be appreciated that the antibody or antigen-binding fragment thereof according to the present invention can be modified using methods well known in the art , e.g., to improve the properties of the antibody or antigen-binding fragment thereof. For example, to slow clearance in vivo and obtain a more desirable pharmacokinetic profile, the antibody or antigen-binding fragment thereof may be modified with polyethylene glycol (PEG). Methods for coupling and site-specifically conjugating PEG to an antibody or antigen-binding fragment thereof are described in, e.g., Leong et al., 2001. Cytokine. 16(3):106-19; Delgado et al., 1996. Br J Cancer. 73(2):175-82. Another non-limitating example of modification consist in the modification of the human Fc region of the antibody in order to enhance their affinity for an Fcγ receptor. Methods of enhancing Fc receptor binding include Fc amino acid modification and modification of Fc carbohydrate structures. For immunoglobulins, it has been demonstrated that the attachment of an N-linked oligosaccharide to Asn-297 of the CH2 domain is critical for ADCC activity. Removal of the N-linked oligosaccharide through mutation of the N-linked consensus site or by enzymatic means results in little or no ADCC activity. Removal of the core a-1,6-fucose moiety from IgG1 Fc oligosaccharides has been demonstrated to improve FcyRIII binding and ADCC activity (see, e.g., Carter, 2001. Nat Rev Cancer. 1(2):118-29; Kanda et al., 2007. Glycobiology. 17(1):104-18; Shields et al., 2002. J Biol Chem. 277(30):26733-40; Shinkawa et al., 2003. J Biol Chem. 278(5):3466-73; Niwa et al., 2004. Cancer Res. 64(6):2127-33). The level of another glycoform, bisected N-linked carbohydrate, has also been suggested to increase ADCC (see, e.g., Umaña et al., 1999. Nat Biotechnol. 17(2):176-80; Hodoniczky et al., 2005. Biotechnol Prog. 21(6):1644-52). A variety of Fc sequence variants with optimized binding affinity for FcyRs and/or enhanced ADCC have been described and are known in the art.

In one embodiment, the antibody or antigen-binding fragment thereof according to the present invention target, block, deplete and/or kill senescent cells to which they are bound.

In one embodiment, the antibody or antigen-binding fragment thereof according to the present invention target, block, deplete and/or kill senescent cells expressing at least one senescent cell-associated antigen, as defined hereinabove.

In one embodiment, the antibody or antigen-binding fragment thereof according to the present invention may comprise human heavy chain constant regions sequences and allow to target, block, deplete and/or kill DEP-1-expressing cells to which they are bound.

In one embodiment, the antibody or antigen-binding fragment thereof according to the present invention may comprise human heavy chain constant regions sequences and allow to target, block, deplete and/or kill DPP4-expressing cells to which they are bound.

In one embodiment, the bispecific antibody or antigen-binding fragment thereof according to the present invention may comprise human heavy chain constant regions sequences and allow to target, block, and/or deplete DEP-1- or DPP4-expressing cells to which they are bound.

In one embodiment, the antibody or antigen-binding fragment thereof according to the present invention depletes and/or kills DEP-1-expressing cells to which they are bound.

In one embodiment, the antibody or antigen-binding fragment thereof according to the present invention depletes and/or kills DPP4-expressing cells to which they are bound.

In one embodiment, the bispecific antibody or antigen-binding fragment thereof according to the present invention depletes and/or kills DEP-1- and DPP4-expressing cells to which they are bound.

The terms “deplete” or “depleting”, with respect to DEP-1- and DPP4-expressing cells refer to the killing, elimination, lysis or induction of such killing, elimination or lysis, so as to negatively affect the number of DEP-1- and DPP4-expressing cells present in a sample or in a subject. In one embodiment, the depletion is via ADCC. In another embodiment, the depletion is via ADCP. In another embodiment, the depletion is via CDC.

Thus, in one embodiment, the antibody of the present invention leads, directly or indirectly, to the depletion of senescent cells, in particular of DEP-1- and/or DPP4-expressing cells (e.g., leads to a 10%, 20%, 50%, 60% or greater elimination or decrease in number of DEP-1- and DPP4-expressing cells).

In one embodiment, the antibody or antigen-binding fragment thereof according to the present invention induces antibody dependent cellular cytotoxicity (ADCC).

The term “antibody-dependent cell-mediated cytotoxicity” or “ADCC” refers to a cell-mediated cytotoxicity induced in an antibody-dependent manner when the Fc region of said antibody bound to its antigen binds to the Fc receptor on effector cells such as natural killer cells, macrophages, neutrophils, eosinophils and mononuclear cells (e.g., peripheral blood mononuclear cells), thereby leading to lysis of the target cell. ADCC can be measured using assays that are known and available in the art (e.g., Clynes et al., 1998. Proc Natl Acad Sci USA. 95(2):652-6).

In one embodiment, the antibody or antigen-binding fragment thereof according to the present invention is from the IgG1 subclass and has ADCC activity.

In one embodiment, the antibody or antigen-binding fragment thereof according to the present invention induces antibody-dependent cell-mediated phagocytosis (ADCP).

The term “antibody-dependent cell-mediated phagocytosis” (ADCP) or “opsonisation” refers to a cell-mediated reaction in which nonspecific cytotoxic cells (e.g., phagocytes, macrophages) that express Fc receptors (FcRs) recognize antibody bound on a target cell and induce phagocytosis of the target cell. ADCP can be measured using assays that are known and available in the art (e.g., Clynes et al., 1998. Proc Natl Acad Sci USA. 95(2):652-6).

In one embodiment, the antibody or antigen-binding fragment thereof according to the present invention is from the IgG1 subclass and has ADCP activity.

In one embodiment, the antibody or antigen-binding fragment thereof according to the present invention induces complement-dependent cytotoxicity (CDC).

The term “complement-dependent cytotoxicity” (CDC) refers to the induction of the lysis of antigen-expressing cells recognized by an antibody or antigen-binding fragment thereof of the invention in the presence of complement. The complement activation pathway is initiated by the binding of the first component of the complement system (C1q) to a molecule (e.g., an antibody) complexed with a cognate antigen. CDC can be measured using assays that are known and available in the art (e.g., Clynes et al., 1998. Proc Natl Acad Sci USA. 95(2):652-6; Gazzano-Santaro et al., 1997. J Immunol Methods. 202(2):163-71).

In one embodiment, the antibody or antigen-binding fragment thereof according to the present invention is from the IgG1 subclass and has CDC activity.

In one embodiment, the antibody or antigen-binding fragment thereof according to the present invention is linked to a toxic moiety.

In one embodiment, the antibody or antigen-binding fragment thereof according to the present invention is not conjugated, such as, for example, to a toxic moiety.

In one embodiment, the antibody or antigen-binding fragment thereof according to the present invention is not linked to a toxic moiety.

The present invention further relates to a nucleic acid sequence encoding the antibody or antigen-binding fragment thereof according to the present invention.

The present invention further relates to a vector that includes the nucleic acid sequence encoding the antibody or antigen-binding fragment thereof according to the present invention.

Also provided herein are methods of manufacture for producing an antibody, or antigen-binding fragment thereof that specifically binds to a senescent cell associated antigen of interest (such, as for example, DEP-1 and/or DPP4). For example, a process (or method) for manufacturing an antibody may comprise determining the nucleotide sequence that encodes the antibody by using standard molecular biology techniques, including primer design, hybridization, nucleic acid isolation, cloning, and amplification, and sequencing. A polynucleotide comprising a nucleotide sequence encoding the antibody, or antigen-binding fragment thereof, may be incorporated into a recombinant expression construct (i.e., vector) according to well-known methods and principles known in the molecular biology art and described herein for preparing a recombinant expression vector.

The nucleic acid molecules encoding the antibody or antigen binding fragment, as described herein, may be propagated and expressed according to any of a variety of routinely practiced procedures for nucleic acid excision, ligation, transformation, and transfection. Thus, in certain embodiments expression of the antibody or antigen binding fragment may be preferred in a prokaryotic host cell, such as Escherichia coli (see, e.g., Plückthun et al., 1989. Methods Enzymol. 178:497-515). In certain other embodiments, the antibody may be expressed in a eukaryotic host cell, including animal cells (including mammalian cells); yeast (e.g., Saccharomyces cerevisiae, Schizosaccharomyces pombe, and Pichia pastoris); or plant cells. Examples of suitable animal cells include, but are not limited to, myeloma, HEK293, COS, or CHO cells.

Examples of plant cells include tobacco, corn, soybean, and rice cells. By methods known to those having ordinary skill in the art and based on the present disclosure, a nucleic acid vector may be designed for expressing foreign sequences in a particular host system, and then polynucleotide sequences encoding the cellular polypeptide may be inserted. The regulatory elements will vary as appropriate for the particular host.

Another object of the invention is a composition comprising, consisting of or consisting essentially of a population of immune cells, preferably a population of isolated immune cells, preferably a population of substantially purified isolated immune cells, engineered to express at the cell surface a CAR or a bispecific CAR according to the invention and as described above.

Another object of the invention is a composition comprising, consisting of or consisting essentially of an isolated bispecific antibody or fragment thereof according to the invention and as described above.

Another object of the invention is a composition comprising, consisting of or consisting essentially of:
at least one isolated and/or substantially purified immune cell population engineered to express at the cell surface a CAR or a bispecific CAR specific for at least one DEP1 and/or DPP4 as described hereinabove,
an isolated bispecific antibody or fragment thereof as described herein above, or
a mixture of an isolated anti-DEP1 antibody or a fragment thereof and an isolated anti-DPP4 antibody or a fragment thereof as described herein above.

In one embodiment, said composition has been frozen and thawed.

In one embodiment, said composition is lyophilized.

In one embodiment, the compositions according to the present invention are pharmaceutical compositions and further comprise at least one pharmaceutically acceptable excipient.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in the pharmaceutical composition of the invention include, without being not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene- polyoxypropylene- block polymers, polyethylene glycol and wool fat.

In one embodiment, the compositions according to the present invention are medicaments.

As used herein, the term “consisting essentially of”, with reference to a pharmaceutical composition or medicament, means that the at least one T cell population of the invention is the only one therapeutic agent or agent with a biologic activity within said pharmaceutical composition or medicament.

Such compositions and medicaments may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Since the present invention selectively target senescent cells, it is contemplated that the compositions according to the present invention may be cosmetic compositions, and further comprise at least one dermatologically acceptable excipient.

The term “dermatologically acceptable excipient” refers to excipient which are suitable for external topical application. Examples of dermatologically acceptable excipients include, but are not limited to, sebum-regulating agents, antibacterial agents, antifungal agents, keratolytic agents, keratoregulating agents, astringents, anti-inflammatory agents, anti-irritants, antioxidants, free-radical scavengers, cicatrizing agents, anti-aging agents and moisturizing agents.

In one embodiment, the composition, pharmaceutical composition, medicament or cosmetic composition of the present invention comprises a combination of immune cell populations as described hereinabove (i.e., at least two distinct immune cell populations of the invention).

In one embodiment, the composition, pharmaceutical composition, medicament or cosmetic composition of the present invention comprises a combination of:
at least one isolated and/or substantially purified immune cell population engineered to express at the cell surface a CAR or a bispecific CAR specific for at least one DEP1 and/or DPP4 as described hereinabove, and/or
an isolated bispecific antibody or fragment thereof as described herein above, and/or
a mixture of an isolated anti-DEP1 antibody or a fragment thereof and an isolated anti-DPP4 antibody or a fragment thereof as described herein above.

The administration of the composition, pharmaceutical composition, medicament or cosmetic composition of the present invention may be carried out in any convenient manner, including by injection, aerosol inhalation, topical delivery (such as, for example, by transdermal delivery), oral delivery, rectal delivery, nasal delivery, or vaginal delivery.

In one embodiment, the composition, pharmaceutical composition, medicament or cosmetic composition of the present invention is in a form adapted for injection, such as, for example, for trans-arterial, intravenous (i.v.), intramuscular, intraperitoneal (i.p.), intrapleural, intradermal, subcutaneous, transdermal injection or infusion.

Examples of forms suitable for injectable use include, but are not limited to, sterile solutions or dispersions and sterile powders for the extemporaneous preparation of sterile injectable solutions or dispersions. The prevention against contamination by microorganisms can be brought about by adding in the composition preservatives such as, for example, various antibacterial and antifungal agents (for example, parabens, chlorobutanol, phenol, sorbic acid, thimerosal and the like). In an embodiment, it may be preferable to include isotonic agents, for example, sugars or sodium chloride, to reduce pain during injection. In one embodiment, prolonged absorption of the injectable compositions can be brought about by the use in the compositions of agents delaying absorption, for example, aluminum monostearate and gelatin.

In one embodiment, the composition, pharmaceutical composition, medicament or cosmetic composition of the present invention is in an adapted form for a parenteral administration. Thus, in one, the composition, pharmaceutical composition, medicament or cosmetic composition of the present invention is to be administered parenterally.

In one embodiment, the composition, pharmaceutical composition, medicament or cosmetic composition of the present invention is in an adapted form for an intravenous administration. Thus, in one embodiment, the composition, pharmaceutical composition, medicament or cosmetic composition of the present invention is to be administered intravenously.

In one embodiment, the composition, pharmaceutical composition, medicament or cosmetic composition of the present invention may be injected directly into the site of the disease or disorder to be treated.

In one embodiment, a lyophilized composition, a lyophilized pharmaceutical composition, a lyophilized medicament or a lyophilized cosmetic composition of the invention is solubilized in water for injection and gently mixed, the mixture is gently mixed and charged into a suitable syringe. This invention thus also relates to a medical device, including a syringe filled or prefilled with a composition, pharmaceutical composition, medicament or cosmetic composition of the invention.

In one embodiment, the composition, pharmaceutical composition, medicament or cosmetic composition of the present invention is formulated for topical administration. Thus, in one embodiment, the composition, pharmaceutical composition, medicament or cosmetic composition is to be administered topically.

Examples of forms adapted for topical administration include, without being limited to, liquid, paste or solid compositions, and more particularly aqueous solutions, drops, dispersions, sprays, microcapsules, micro- or nanoparticles, polymeric patch, or controlled-release patch, and the like.

The present invention further relates to composition and pharmaceutical compositions as described herein above, for use as a medicament.

The present invention further relates to composition, pharmaceutical composition or medicament as described herein, for use in treating, preventing or alleviating senescence-related diseases or disorders.

The present invention thus further relates to a method for treating, preventing or alleviating senescence-related diseases or disorders in a subject, comprising administering to the subject the composition, pharmaceutical composition or medicament of the invention.

As used herein, the term “senescence-associated”, “senescence-related” or “age-related” diseases, disorders, or conditions refers to a physiological condition that presents with one or more symptoms or signs, wherein a subject having the condition needs or would benefit from a lessening of such symptoms or signs. The condition is senescence-associated if it is caused or mediated in part by senescent cells, which may be induced by multiple etiologic factors including age, DNA damage, oxidative stress, genetic defects, etc. Lists of senescence-associated disorders that can potentially be treated or managed using the methods and products taught in this disclosure include those discussed in this disclosure and the previous disclosures to which this application claims priority.

Non-limiting examples of senescence-related diseases include : fibrotic diseases, chronic inflammatory diseases (e.g., arthritis or arthrosis), cancer, premalignant disorders, atherosclerosis, osteoarthritis, diabetes, diabetic ulcers, kyphosis, scoleosis, hepatic insufficiency, cirrhosis, Hutchinson-Gilford progeria syndrome (HGPS), laminopaties, osteoporosis, dementia, (cardio)vascular diseases (e.g., angina, arrhythmia, atherosclerosis, cardiomyopathy, congestive heart failure, coronary artery disease (CAD), carotid artery disease, endocarditis, heart attack, coronary thrombosis, myocardial infarction, high blood pressure/hypertension, hypercholesterolemia/hyperlipidemia, mitral valve prolapsed, peripheral artery disease (PAD) and stroke), obesity, metabolic syndrome, acute myocardial infarction, emphysema, insulin sensitivity, boutonneuse fever, sarcopenia, neurodegenerative diseases (e.g., Alzheimer's, Huntington's or Parkinson's disease), cataract, anemia, hypertension, age-related macular degeneration, COPD, asthma, renal insufficiency, incontinence, hearing loss such as deafness, vision loss such as blindness, sleeping disturbances, pain such as joint pain or leg pain, imbalance, fear, depression, breathlessness, weight loss, hair loss, muscle loss, loss of bone density, frailty and/or reduced fitness.

The present invention thus further relates to composition, pharmaceutical composition or medicament as described herein for use in the treatment of fibrotic diseases, premalignant disorders inflammatory diseases and cancers.

The present invention thus further relates to a method for treating fibrotic diseases, premalignant disorders inflammatory diseases and cancers in a subject, comprising administering to the subject the composition, pharmaceutical composition or medicament of the invention.

Senescent cells are present in fibrosis of many tissues including but not limited to skin, liver, lung, pancreas and prostate. Thus, the present inventors contemplate treating fibrotic diseases of such tissues. Exemplary fibrotic diseases which may be treated by the invention include but are not limited to eosinophilic esophagitis, hypereosinophilic syndromes (HES), Loeffler's endomyocarditis, endomyocardial fibrosis, idiopathic pulmonary fibrosis, and scleroderma.

In one embodiment, the pulmonary fibrotic disease to be treated, prevented or alleviated is selected from the group comprising idiopathic pulmonary fibrosis (IPF), chronic obstructive pulmonary disease (COPD) acute respiratory distress syndrome (ARDS), combined pulmonary fibrosis and emphysema (CPFR), pulmonary edema, Löffler's syndrome, eosinophilic pneumonia, respiratory hypersensitivity, allergic bronchopulmonary aspergillosis (ABPA), Hamman-Rich syndrome, sarcoidosis, pneumoconiosis, and hypersensitivity pneumonitis (HP).

In one embodiment, the pulmonary fibrotic disease to be treated, prevented or alleviated is chronic obstructive pulmonary disease (COPD) or idiopathic pulmonary fibrosis.

As used herein, the phrase “premalignant lesion” refers to a mass of cells and/or tissue having increased probability of transforming into a malignant tumor. Examples of pre-malignant lesions include, but are not limited to, adenomatous polyps, Barrett's esophagus, Pancreatic Intraepithelial Neoplasia (PanIN), IPMN (Intraductal Papillary Mucinus Neoplasia), DCIS (Ductal Carcinoma in Situ) in the breast, leukoplakia and erythroplakia. Thus, the premalignant lesion to be treated by the invention can transform into a malignant solid or non-solid (e.g., hematological malignancies) cancer (or tumor). According to one embodiment, the premalignant lesion which to be treated is an adenomatous polyp of the colon, an adenomatous polyp of the rectum, an adenomatous polyp of the small bowel and Barrett's esophagus.

As used herein, the term “inflammatory diseases” refers to any abnormality associated with inflammation, such as, for example, chronic inflammatory diseases, acute inflammatory diseases. Examples of inflammatory disorders include but are not limited to, rheumatic diseases, neurological diseases, cardiovascular diseases, uro-gynecological diseases, eye and ear diseases, mucocutaneous diseases, infectious diseases, graft rejection diseases and allergic diseases.

Examples of rheumatic diseases include, but are not limited to, arthritis, osteoarthritis, rheumatoid arthritis, osteoporosis, fibromyalgia, lupus, systemic lupus erythematosus and scleroderma.

Examples of neurological diseases include, but are not limited to, multiple sclerosis, Alzheimer's disease, Parkinson's disease, Huntington's disease, stroke, traumatic brain injury, spinal cord injury, dystonia, chronic regional pain syndrome, motor neuron disease/amyotrophic lateral sclerosis, Guillain-Barre syndrome, muscular dystrophy, cerebral palsy, neuropathy and myositis.

Examples of cardiovascular diseases include, but are not limited to, coronary heart disease, stroke, hypertensive heart disease, inflammatory heart disease, rheumatic heart disease, aortic aneurysm and dissection, congenital heart disease, deep vein thrombosis and pulmonary embolism and atherosclerosis.

Examples of uro-gynecological diseases include, but are not limited to, glomerulonephritis, urinary incontinence and prolapse.

Examples of uro-gynecological diseases include, but are not limited to, cataract, glaucoma, age-related macular degeneration (AMD), presbyopia, dry eyes, corneal diseases, diabetic retinopathy, vertigo, tinnitus and Meniere's disease.

Examples of mucocutaneous diseases include, but are not limited to, eczema, xeroderma pigmentosum, oral lichen planus, mucous membrane pemphigoid and pemphigus vulgaris.

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Examples of diseases associated with transplantation of a graft include, but are not limited to, graft rejection, chronic graft rejection, subacute graft rejection, hyperacute graft rejection, acute graft rejection and graft versus host disease.

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

The present invention further relates to composition, pharmaceutical composition or medicament as described herein, for use in the treatment of a cancerous disease.

The present invention thus further relates to a method for treating cancers in a subject, comprising administering to the subject the composition, pharmaceutical composition or medicament of the invention.

Non-limiting examples of cancers which may be treated according to this aspect of the present invention include: adenocarcinoma, adrenal gland tumor, ameloblastoma, anaplastic, anaplastic carcinoma of the thyroid, angiofibroma, angioma, angiosarcoma, apudoma, argentaffmoma, arrhenoblastoma, ascites tumor cell, ascitic tumor, astroblastoma, astrocytoma, ataxia-telangiectasia, atrial myxoma, a basal cell carcinoma cell, bone cancer, brainstem glioma, brain tumor, breast cancer, Burkitt's lymphoma, cerebellar astrocytoma, cervical cancer, cherry angioma, cholangiocarcinoma, cholangioma, chondroblastoma, chondroma, chondrosarcoma, chorioblastoma, choriocarcinoma, colon cancer, common acute lymphoblastic leukemia, craniopharyngioma, cystocarcinoma, cystofbroma, cystoma, ductal carcinoma in situ, ductal papilloma, dysgerminoma, encephaloma, endometrial carcinoma, endothelioma, ependymoma, epithelioma, erythroleukemia, Ewing's sarcoma, extra nodal lymphoma, feline sarcoma, fibro adenoma, fibro sarcoma, follicular cancer of the thyroid, ganglioglioma, gastrinoma cell, glioblastoma multiform, glioma, gonadoblastoma, haemangioblastoma, haemangioendothelioblastoma, haemangioendotheli oma, haemangiopericytoma, haematolymphangioma, haemocytoblastoma, haemocytoma, hairy cell leukemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angioforoma, Kaposi sarcoma, kidney tumor, large cell lymphoma, leukemia, a leukemia, acute leukemia, lipoma, liver cancer, liver metastases, Lucke carcinoma, lymphadenoma, lymphangioma, lymphocytic leukemia, lymphocytic lymphoma, lymphoeytoma, lymphoedema, lymphoma, lung cancer, malignant mesothelioma, malignant teratoma, mastocytoma, medulloblastome., melanoma, meningioma, mesothelioma, Morton's neuroma, multiple myeloma, myeloblastoma, myeloid leukemia, myelolipoma, myeloma, myoblastoma, myxoma, nasopharyngeal carcinoma, neoplastic, nephroblastoma, neuroblastoma, neurofibroma, neurofibromatosi s, neuroglioma, neuroma, non-Hodgkin's lymphoma, oligodendroglioma, optic glioma, osteochondroma, osteogenic sarcoma, osteosarcoma, ovarian cancer, Paget's disease of the nipple, pancoast tumor, pancreatic cancer, phaeochromocytoma, pheoehromocytoma, plasmacytoma, primary brain tumor, progonoma, prolactinoma, renal cell carcinoma, retinoblastoma, rhabdomyosarcoma, rhabdo sarcoma, a solid tumor, sarcoma, a secondary tumor, seminoma, skin cancer, small cell carcinoma, squamous cell carcinoma, strawberry haemangioma, T-cell lymphoma, teratoma, testicular cancer, thymoma, trophoblastic tumor, Wilm's tumor.

In one embodiment, the composition, pharmaceutical composition or medicament as described hereinabove is used alone.

In another embodiment, the combination, pharmaceutical combination, medicament as described hereinabove is used in combination with at least one anti-cancer agents.

Indeed, many of existing and potential anti-cancer agents induce senescence of cancer cells, therefore the present invention can be used in combination with these agents to increase the efficacy of the anti-cancer treatment. Treatment by these agents can also reduce side effects of radiotherapy or chemotherapy with DNA-damaging agents.

Thus, the combination, pharmaceutical combination or medicament as described hereinabove can be used as an adjuvant therapy along with other treatment modalities for cancers, which are selected based on cancer type, location, the cell type and the grade of malignancy. Conventional therapies include surgery, radiation therapy, and chemotherapy.

Exemplary anti-cancer drugs that can be co-administered with the agents of the invention include, but are not limited to acivicin, aclarubicin, acodazole hydrochloride, acronine, adriamycin, adozelesin, aldesleukin, altretamine, ambomycin, ametantrone acetate, aminoglutethimide, amsacrine, anastrozole, anthramycin, asparaginase, asperlin, azacytidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesyl ate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interferon alfa-2a, interferon alfa-2b, interferon alfa-n1, interferon alfa-n3, interferon beta-1a, interferon gamma-1b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, tali somycin, taxol, tecogal an sodium, tegafur, teloxantrone hydrochloride, temoporfin, teniposide, teroxirone, testolactone, thiamiprine, thioguanine, thiotepa, tiazofuirin, tirapazamine, topotecan hydrochloride, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, and zorubicin hydrochloride.

Additional antineoplastic agents include those disclosed in Chabner et al., 2001.“Antineoplastic agents”. In Goodman et al. (Eds.), Goodman & Gilman's The pharmacological basis of therapeutics (10^{th} ed., pp. 1315-1404). New York, N.Y.: McGraw-Hill, which are incorporated by reference.

The present invention further relates to the composition, pharmaceutical composition, medicament or cosmetic composition as described herein, for cosmetic use.

Indeed, since the present invention selectively target senescent cells, it is contemplated that the present invention could be used for skin care, skin anti-aging and/or skin rejuvenation.

The present invention thus further relates to a method for rejuvenating the skin in a subject, comprising administering to the subject the composition, pharmaceutical composition, medicament or cosmetic composition of the invention.

Because senescent cells drive age-related pathologies, a selective elimination of these cells can prevent or delay age-related deterioration. Thus, senescent cells may be therapeutic targets in the treatment of aging and age-related disease. As such, removal of senescent cells may delay tissue dysfunction and extend health span. Clearance of senescent cells is expected to improve tissue milieu, thereby improving the function of the remaining non-senescent cells.

The present invention further relates to composition, pharmaceutical composition or medicament as described herein, for depleting and/or killing senescent cells.

The present invention thus further relates to a method for depleting and/or killing senescent cells in a subject, comprising administering to the subject the composition, pharmaceutical composition or medicament of the invention.

In one embodiment, the at least one isolated and/or substantially purified immune cell population engineered to express at the cell surface a CAR or a bispecific CAR as described hereinabove, composition, pharmaceutical composition or medicament, according to the present invention is to be administered to the subject in need thereof in a therapeutically effective amount.

In one embodiment, the isolated antibody or antigen-binding fragment thereof, nucleic acid, expression vector, composition, pharmaceutical composition or medicament according to the present invention is to be administered to the subject in need thereof in a therapeutically effective amount.

It will be however understood that the total daily usage of the immune cells population as described herein, isolated antibody or antigen-binding fragment thereof, nucleic acid, expression vector, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the isolated antibody or antigen-binding fragment thereof, nucleic acid, expression vector, composition, pharmaceutical composition or medicament employed; the age, body weight, general health, sex and diet of the subject; the time of administration, route of administration, and rate of excretion of the specific isolated antibody or antigen-binding fragment thereof, nucleic acid, expression vector, composition, pharmaceutical composition or medicament employed; the duration of the treatment; drugs used in combination or coincidental with the specific isolated antibody or antigen-binding fragment thereof, nucleic acid, expression vector, composition, pharmaceutical composition or medicament employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

For example, regimens or dosages used for administration of the immune cells population of the invention or the antibody of the invention can be adapted as function of various parameters, and in particular as a function of the mode of administration used, of the relevant pathology, or of the desired duration of treatment. For example, it is well within the skill of the art to start doses of the antibodies at levels lower than those required to achieve the desired therapeutic effect and to gradually increase the dosage until the desired effect is achieved.

### BRIEF DESCRIPTION OF THE DRAWINGS

FIG. 1—‘The structure of CAR vector’—is a schema illustrating the construction of the full-length CAR expression cassette, subcloned into Lenti-EF1a-rhuMAB 41-VH-Linker-VL-CFPCART, pCDCAR1. The full length of chimeric antigen receptor was synthesized and subcloned into lentivirus vector. The insert was confirmed by Sanger sequencing.

FIG. 2—‘The ADCC Assay’—is a graph showing the relative toxicity in young and old fibroblast cells (WI38 cells), using 0.05 μg/mL or 0.5 μg/ml of anti-DPP4 antibody.

FIG. 3—‘The CAR-T assay’—is a graph showing the results of selective elimination of senescent cells by engineered CAR-T against DPP4.

### EXAMPLES

The present invention is further illustrated by the following examples.

Materials and Methods

Expression of DPP4 (rhuMAB 41) Antibody and Antigen

The full-length of the antigen was synthesized and subcloned into an expression vector. The insert was confirmed by Sanger sequencing. After the vector was verified by sequencing, the vector was expressed in CHO-S cell line with chemically defined culture media. After 9 days cultivation, the protein was purified by Nickel columns, ultrafiltration and then subjected to 0.2-micron sterile filtration to get the bulk of high purity.

The heavy chain and light chain of the rhuMAB 41 antibody (Creative Biolabs) were synthesized and subcloned into CBL property expression vector, respectively. The insert was confirmed by Sanger sequencing. After the vectors were verified by sequencing, the vectors were expressed in HEK293 cell line with chemically defined culture media. After 9 days cultivation, the protein was purified by Protein A affinity chromatography, ultrafiltration and then subjected to 0.2-micron sterile filtration to get the bulk of high purity.

Expression of DPP4 (rhuMAB 41) scFv

The scFv consists of variable regions of heavy and light chains that are joined together by a flexible peptide linker. In the scFv, the order of the domains can be either VH-linker-VL or VL-linker-VH. The affinity of the two construction types to the target might be different. Hence, the two construction types can lead to secretory expression in different level.

The scFv(s) were expressed and tested by flow cytometry to evaluate the binding affinity to target cells. 5×10⁵ WI-38 cells were co-cultured with rhuMAB 41 antibody (humanized antibody), VL-Linker-VH antibody and VH-Linker-VL antibody (1 μg/tube), respectively, and then analyzed by using PE-anti-Human IgG Fc as secondary antibody.

The results indicated that VH-linker-VL antibody has higher affinity for target cell WI-38 and was chosen for CAR development.

CAR-T Cell Preparation and Construction

Primary human T cells were used for CAR-T generation. Human primary T cells were isolated from PBMCs of healthy donors by magnetic beads and stimulated in growth medium supplemented with IL-2. Activated T cells were then transduced with lentivirus expressing customized chimeric antigen receptor. After CAR-T cell expansion, CAR-transduction efficiency was examined by FACS and qPCR.

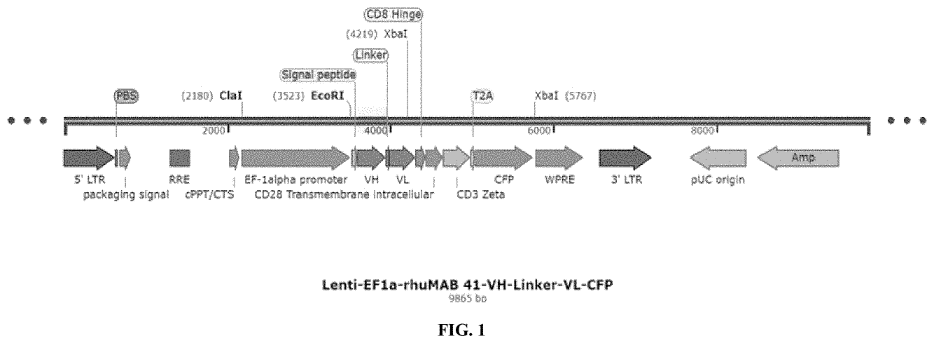

The full-length of chimeric antigen receptor was synthesized and subcloned into lentivirus vector. The insert was confirmed by Sanger sequencing. The structure of CAR vector is schematically illustrated in FIG. 1.

Lentiviral vectors, which were derived from immunodeficiency viruses, were used for their relatively high efficiency for T cell transduction and their ability of infection of the non-proliferating cells. A second generation of the packaging system was utilized to generate transduction-ready pseudoviral particles in HEK293T cells. The titer of the lentivirus particle was determined by qPCR and cell-based titration assay. The results of lentivirus titration showed that prepared virus stock was at a high titer of 3.27×10⁸ TU/mL.

Primary Human T lymphocytes Preparation and CAR-T Preparation

PBMC from a healthy human donor was stimulated with anti-CD3/CD28 magnetic beads and the T cells were isolated using magnetic cell separation system. To generate CAR-T cells, the lentivirus particles with DPP4-CAR coding gene were incubated with the T cells in the presence of polybrene. After T cell expansion, the CAR-T cells were used for in vitro cytotoxicity assays.

Target Cell Preparation

WI38 target cells were obtained at population doubling 19 (PD19) and passaged until they stopped proliferating. They were further analyzed by FACS for the detection of their surface antigen DPP4.

Natural killer (NK) cell preparation

Effector cells (NK cells) were freshly prepared before the ADCC assay. By using EasySep™ Human NK Cell Isolation Kit (STEMCELL, Catalog:17955), NK cells were isolated from a healthy human donor and resuspended in RPMI 1640 medium at 5×10⁶/mL.

ADCC Assay

The target cells (WI38), i.e., the senescent cells and proliferating cells, were plated into a 24-well plate at 1×10⁵ cell/well in 100 μL RPMI-1640 supplied with 5% FBS 24 hour and cultured overnight. On the day of the assay, anti-DPP4 antibody (rhuMAB 41) was added into each well at a final concentration of 0 μg/mL, 0.05 μg/mL, and 0.5 μg/mL. After 30 minutes of incubation at 37° C. with 5% CO₂, 100 μL NK cells (5×10⁵ cell/well) were added to each well at E/T=5:1. After incubation for 6 hours at 37° C. with 5% CO₂, the cells were stained with eBioscience™ Annexin V Apoptosis Detection Kit PE (Invitrogen, Catalog: 88-8102-74) with 7-AAD and analyzed by flow cytometry.

In vitro CAR-T Activity Assay by FACS

In vitro analysis was used for evaluation of the targeting effect of DPP4-CAR-T. The target cells (WI38), i.e., the senescent cells and proliferating cells, were plated into a 24-well plate at 1×10⁵ cell/well in 100 μL RPMI-1640 supplied with 5% FBS and cultured overnight. On the day of the assay, effector cells (5×10⁵ cell/well) were added to each well at E/T=5:1. After incubation for 6 hours at 37 C.° with 5% CO₂, the cells were stained with eBioscience™ Annexin V Apoptosis Detection Kit PE (Invitrogen, Catalog: 88-8102-74) with 7-AAD and analyzed by flow cytometry. The relative percentage was quantified by normalizing the results to control CAR-T.

Results

ADCC Assay

In vitro Antibody-Dependent Cell-Mediated Cytotoxicity (ADCC) assays are common tools for immunotherapeutic drug discovery and biosimilar development. For this experiment, cytotoxicity is defined as apoptosis monitored by annexin-V positive and 7-ADD negative cells counted.

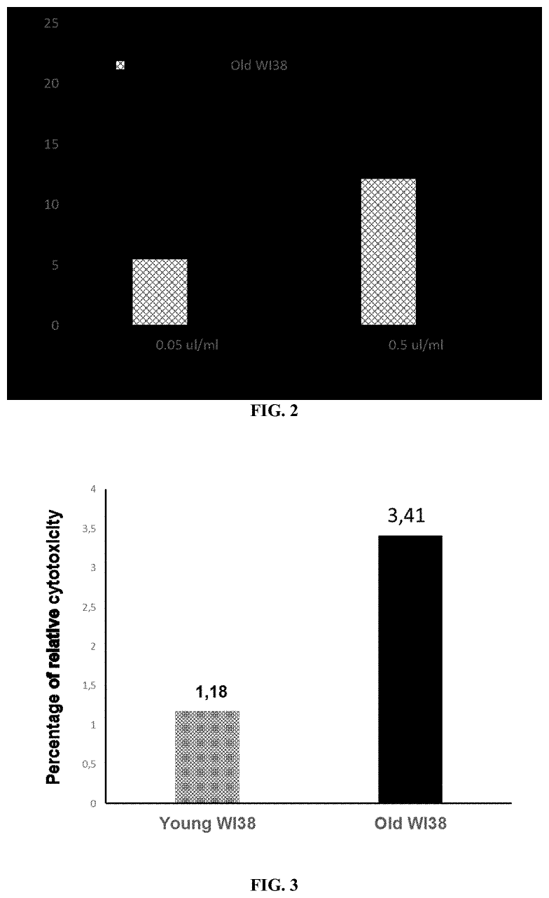

Results of a standard ADCC assay are shown in FIG. 2. This experiment depicts a difference between young and old fibroblast cells at indicated antibody concentration as listed 1.5 and 1.65-fold, respectively.

Our results show that high abundance of DPP4 on the surface of senescent cells lead to a 1.5-fold selective elimination of senescent cells using anti-DPP4 antibody under optimal conditions.

DPP4-CAR-T Assay

When we used the same cells with control and DPP4-CAR-T cells, we observe a 3-fold selective elimination of old senescent cells with respect to the young ones. CAR-T cells engineered against DPP4 show high specificity against DPP4-bearing senescent cells (FIG. 3).

1. A chimeric antigen receptor (CAR) comprising:
(i) at least one extracellular binding domain, comprising at least one antigen-binding domain directed to a senescent cell-associated antigen, preferably to DEP1 and/or DPP4,
(ii) an extracellular spacer domain,
(iii) a transmembrane domain,
(iv) optionally at least one costimulatory domain, and
(v) at least one intracellular signaling domain.

2. The CAR according to claim 1, wherein said at least one antigen-binding domain is directed to DEP1.

3. The CAR according to claim 1, wherein said at least one antigen-binding domain is directed to DPP4.

4. The CAR according to claim 1, wherein said CAR is a bispecific CAR comprising two antigen-binding domains.

5. The CAR according to claim 4, wherein each of the at least two antigen-binding domains binds to a different antigen.

6. The CAR according to claim 4, wherein each of the at least two antigen-binding domains binds to DEP1 and DPP4.

7. An isolated immune cell population expressing at least one CAR according to claim 1.

8. The isolated immune cell population according to claim 7, wherein said isolated immune cell population expresses at least one CAR according to claim 2 and at least one CAR according to claim 3.

9. The isolated immune cell population according to claim 7, wherein said isolated immune cell population expresses at least one CAR according to claim 4.

10. The isolated immune cell population according to claim 7, comprising immune cells selected from the group consisting of T cells, natural killer (NK) cells, or a combination thereof.

11. An isolated bispecific antibody or a fragment thereof, comprising at least two antigen binding domains directed to at least two senescent cell-associated antigens, preferably the at least two senescent cell-associated antigens are DEP1 and DPP4.

12. The isolated bispecific antibody or fragment thereof according to claim 11, comprising:
(i) an antigen-binding domain of an anti-human DEP1 antibody or a fragment thereof; and
(ii) an antigen-binding domain of an anti-human DPP4 antibody or a fragment thereof.

13. A composition comprising:
the isolated immune cell population according to claim 7;
the isolated bispecific antibody or fragment thereof according to claim 11; or
a mixture of an isolated anti-human DEP1 antibody or a fragment thereof and an isolated anti-human DPP4 antibody or a fragment thereof.

14. The composition according to claim 13, being a pharmaceutical composition and further comprising at least one pharmaceutically acceptable excipient.

15. A method for treating, preventing or alleviating senescence-related diseases or disorders in a subject in need thereof, comprising administering to said subject the composition according to claim 13.

16. The method according to claim 15, wherein senescence-related diseases or disorders are selected from the group consisting of fibrotic diseases, premalignant disorders, inflammatory diseases and cancers.

17. A method for depleting and/or killing senescent cells in a subject in need thereof, comprising administering to said subject the composition according to claim 13.
